(19) United States
(12) Patent Application Publication
(10) Pub. No.: US 2004/0058915 A1
(43) Pub. Date: Mar. 25, 2004

(54) Compounds containing a bicyclic ring system useful as alpha v beta 3 antagonists

(76) Inventors: Ish Kumar Khanna, Libertyville, IL (US); Yi Yu, Glenview, IL (US); Devadas Balekudra, Chesterfield, MO (US); Hwang-Fun Lu, Ballwin, MO (US); Nizal S. Chandrakumar, Vernon Hills, IL (US)

Correspondence Address: Pharmacia Corporation, Global Patent Department, Mail Zone MC5, PO Box 1027, St Louis, MO, 63141, US

(21) Appl. No.: 10/363,070

(22) PCT Filed: Aug. 29, 2001
(86) PCT No.: PCT/US01/26889

(51) Int. Cl. A61K 31/5415; A61K 31/538; A61K 31/517; A61K 31/502; A61K 31/498; A61K 31/47; A61K 31/428; A61K 31/423; A61K 31/4184

(52) U.S. Cl. 514/224.2; 514/230.5; 514/248; 514/249; 514/266.1; 514/307; 514/311; 514/367; 514/394; 514/406; 514/415; 514/375; 544/105; 544/51; 544/283; 544/235; 544/353; 546/176; 548/304.4; 548/152; 548/217; 548/495

(57) ABSTRACT

The present invention relates to a class of compounds represented by the Formula I or a pharmaceutically acceptable salt thereof, pharmaceutical compositions comprising compounds of the Formula I, and methods of selectively inhibiting or antagonizing the αvβ₃ and/or αvβ₅ integrin. The ring A-B, is selected from the group consisting of the formula II all optionally substituted and bonded to X and Z₁ at any position.

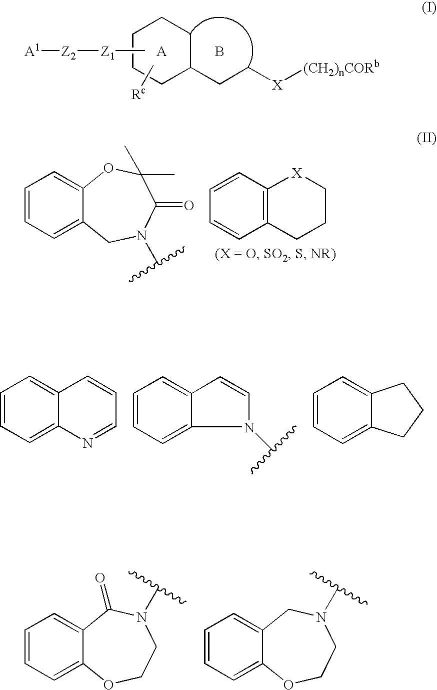

The present application claims priority under Title 35, United States Code, §119 of U.S. Provisional application Serial No. 60/228,693 filed Aug. 29, 2000.

### FIELD OF THE INVENTION

The present invention relates to pharmaceutical agents which are α_{V}β₃ and/or α_{V}β₅ integrin antagonists and as such are useful in pharmaceutical compositions and in methods for treating conditions mediated by α_{V}β₃ and/or α_{V}β₅ integrins.

### BACKGROUND OF THE INVENTION

Integrins are a group of cell surface glycoproteins which mediate cell adhesion and therefore are useful mediators of cell adhesion interactions which occur during various biological processes. Integrins are heterodimers composed of noncovalently linked α and β polypeptide subunits. Currently eleven different α subunits have been identified and six different β subunits have been identified. The various α subunits can combine with various β subunits to form distinct integrins.

The integrin identified as α_{V}β₃ (also known as the vitronectin receptor) has been identified as an integrin which plays a role in various conditions or disease states including tumor metastasis, solid tumor growth (neoplasia), osteoporosis (Ross, et al., J. Biol, Chem., 1987, 262, 7703), Paget's disease, humoral hypercalcemia of malignancy (Carron et al., Cancer Res. 1998, 58, 1930), osteopenia (Lark et al., J Bone Miner Res. 2001,16, 319), endometriosis (Healy et al., Hum. Reproductive Update, 1998, 4, 736), angiogenesis, including tumor angiogenesis (Cheresh, Cancer Metastasis Rev., 1991, 10, 3-10 and Brooks, et al., Cell, 1994, 79, 1157), retinopathy including macular degeneration (Friedlander et al., Proc. Natl. Acad. Sci USA 1996, 93, 9764), arthritis, including rheumatoid arthritis (Badger et al., Arthritis Rheum, 2001, 44, 128), periodontal disease, psoriasis and smooth muscle cell migration (e.g. restenosis and artherosclerosis, (Brown et al., Cardiovascular Res., 1994, 28, 1815). The compounds of the present invention are α_{V}β₃ antagonists and can be used, alone or in combination with other therapeutic agents, in the treatment or modulation of various conditions or disease states described above. Additionally, it has been found that such agents would be useful as antivirals, antifungals and antimicrobials. Thus, compounds which selectively antagonize α_{V}β₃ would be beneficial for treating such conditions.

The integrin α_{V}β₅ plays a role in neovascularization. Antagonists of the α_{V}β₅ integrin will inhibit neovascularization and will be useful for treating and preventing angiogenesis metastasis, tumor growth, macular degeneration and diabetic retionopathy. M. C. Friedlander, et al., Science, 270, 1500-1502 (1995) disclose that a monoclonal antibody for α_{V}β₅ inhibits VEFG-induced angogenesis in the rabbit cornea and the chick chorioallantoic membrane model. Therefore, it would be useful to antagonize both the α_{V}β₅ and the α_{V}β₃ receptor. Such “mixed α_{V}β₅/α_{V}β₃ antagonists” or “dual α_{V}β₃/α_{V}β₅ antagonists” would be useful for treating or preventing angiogenesis, tumor metastasis, tumor growth, diabetic retinopathy, macular degeneration, atherosclerosis and osteoporosis.

It has been shown that the α_{V}β₃ integrin and other α_{V} containing integrins bind to a number of Arg-Gly-Asp (RGD) containing matrix macromolecules. Compounds containing the RGD sequence mimic extracellular matrix ligands so as to bind to cell surface receptors. However, it is also known that RGD peptides in general are nonselective for RGD dependent integrins. For example, most RGD peptides which bind to α_{V}β₃ also bind to α_{V}β₅, α_{V}β₁ and α_{IIb}β₃. Antagonism of platelet α_{IIb}β₃ (also known as the fibrinogen receptor) is known to block platelet aggregation in humans. In order to avoid bleeding side-effects when treating the conditions or disease states associated with the integrin α_{V}β₃, it would be beneficial to develop compounds which are selective antagonists of α_{V}β₃ as opposed to α_{IIb}β₃.

Tumor cell invasion occurs by a three step process: 1) tumor cell attachment to extracellular matrix; 2) proteolytic dissolution of the matrix; and 3) movement of the cells through the dissolved barrier. This process can occur repeatedly and can result in metastases at sites distant from the original tumor.

Seftor et al. (Proc. Natl. Acad. Sci. USA, Vol. 89 (1992) 1557-1561) have shown that the α_{V}β₃ integrin has a biological function in melanoma cell invasion. Montgomery et al., (Proc. Natl. Acad. Sci. USA, Vol. 91 (1994) 8856-60) have demonstrated that the integrin α_{V}β₃ expressed on human melanoma cells promotes a survival signal, protecting the cells from apoptosis. Mediation of the tumor cell metastatic pathway by interference with the α_{V}β₃ integrin cell adhesion receptor to impede tumor metastasis would be beneficial.

Brooks et al. (Cell, Vol. 79 (1994) 1157-1164) have demonstrated that antagonists of α_{V}β₃ provide a therapeutic approach for the treatment of neoplasia (inhibition of solid tumor growth) since systemic administration of α_{V}β₃ antagonists causes dramatic regression of various histologically distinct human tumors.

The adhesion receptor integrin α_{V}β₃ was identified as a marker of angiogenic blood vessels in chick and man and therefore such receptor plays a critical role in angiogenesis or neovascularization. Angiogenesis is characterized by the invasion, migration and proliferation of smooth muscle and endothelial cells. Antagonists of α_{V}β₃ inhibit this process by selectively promoting apoptosis of cells in neovasculature. The growth of new blood vessels, or angiogenesis, also contributes to pathological conditions such as diabetic retinopathy including macular degeneration (Adamis et al., Amer. J. Ophthal., Vol. 118, (1994) 445-450) and rheumatoid arthritis (Peacock et al., J. Exp. Med., Vol. 175, (1992), 1135-1138). Therefore, α_{V}β₃ antagonists would be useful therapeutic agents for treating such conditions associated with neovascularization (Brooks et al., Science, Vol. 264, (1994), 569-571).

It has been reported that the cell surface receptor α_{V}β₃ is the major integrin on osteoclasts responsible for attachment to bone. Osteoclasts cause bone resorption and when such bone resorbing activity exceeds bone forming activity it results in osteoporosis (loss of bone), which leads to an increased number of bone fractures, incapacitation and increased mortality. Antagonists of α_{V}β₃ have been shown to be potent inhibitors of osteoclastic activity both in vitro [Sato et al., J. Cell. Biol., Vol. 111 (1990) 1713-1723] and in vivo [Fisher et al., Endocrinology, Vol. 132 (1993) 1411-1413]. Antagonism of α_{V}β₃ leads to decreased bone resorption and therefore restores a normal balance of bone forming and resorbing activity. Thus it would be beneficial to provide antagonists of osteoclast α_{V}β₃ which are effective inhibitors of bone resorption and therefore are useful in the treatment or prevention of osteoporosis.

The role of the α_{V}β₃ integrin in smooth muscle cell migration also makes it a therapeutic target for prevention or inhibition of neointimal hyperplasia which is a leading cause of restenosis after vascular procedures (Choi et al., J. Vasc. Surg. Vol. 19(1) (1994) 125-34). Prevention or inhibition of neointimal hyperplasia by pharmaceutical agents to prevent or inhibit restenosis would be beneficial.

White (Current Biology, Vol. 3(9)(1993) 596-599) has reported that adenovirus uses α_{V}β₃ for entering host cells. The integrin appears to be required for endocytosis of the virus particle and may be required for penetration of the viral genome into the host cell cytoplasm. Thus compounds which inhibit α_{V}β₃ would find usefulness as antiviral agents.

### SUMMARY OF THE INVENTION

The compounds of this invention are 1) α_{V}β₃ integrin antagonists; or 2) α_{V}β₅ integrin antagonists; or 3) mixed or dual α_{V}β₃/α_{V}β₅ antagonists. The present invention includes compounds which inhibit the respective integrins and also includes pharmaceutical compositions comprising such compounds. The present invention further provides for methods for treating or preventing conditions mediated by the α_{V}β₃ and/or α_{V}β₅ receptors in a mammal in need of such treatment comprising administering a therapeutically effective amount of the compounds of the present invention and pharmaceutical compositions of the present invention. Administration of such compounds and compositions of the present invention inhibits angiogenesis, tumor metastasis, tumor growth, osteoporosis, Paget's disease, humoral hypercalcemia of malignancy, retinopathy, macular degeneration, arthritis, periodontal disease, smooth muscle cell migration, including restenosis and artherosclerosis, and viral diseases.

The present invention relates to a class of compounds represented by the Formula I

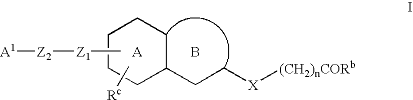

or a pharmaceutically acceptable salt thereof, wherein

A¹ is a 5-9 membered monocyclic or 7-12 membered bicyclic heterocycle of the formula

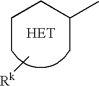

containing at least one nitrogen atom and optionally 1 to 3 heteroatoms, selected from the group consisting of O, N or S; optionally saturated or unsaturated; optionally substituted by one or more R^{k} selected from the group consisting of hydroxy, alkyl, cycloalkyl, alkoxy, alkoxyalkyl, thioalkyl, cyano, amino, alkylamino, halogen, acylamino, sulfonamide and —COR wherein R is hydroxy, alkoxy, alkyl or amino;

or or A¹ is

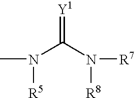

wherein Y¹ is selected from the group consisting of N—R², O, and S;

R² is selected from the group consisting of H; alkyl; cycloalkyl; aryl; hydroxy; alkoxy; cyano; alkenyl; alkynyl; amido; alkylcarbonyl; arylcarbonyl; alkoxycarbonyl; aryloxycarbonyl; haloalkylcarbonyl; haloalkoxycarbonyl; alkylthiocarbonyl; arylthiocarbonyl; acyloxymethoxycarbonyl;

R² taken together with R⁷ forms a 4-12 membered dinitrogen containing heterocycle optionally substituted with one or more substituent selected from the group consisting of lower alkyl, thioalkyl, alkylamino, hydroxy, keto, alkoxy, halo, phenyl, amino, carboxyl or carboxyl ester, and fused phenyl;

or R² taken together with R⁷ forms a 4-12 membered heterocycle containing one or more heteroatom selected from O, N and S optionally unsaturated;

or R² taken together with R⁷ forms a 5 membered heteroaromatic ring fused with a aryl or heteroaryl ring;

R⁷ (when not taken together with R²) and R⁸ are independently selected from the group consisting of H; alkyl; alkenyl; alkynyl; aralkyl; amino; alkylamino; hydroxy; alkoxy; arylamino; amido, alkylcarbonyl, arylcarbonyl; alkoxycarbonyl; aryloxy; aryloxycarbonyl; haloalkylcarbonyl; haloalkoxycarbonyl; alkylthiocarbonyl; arylthiocarbonyl; acyloxymethoxycarbonyl; cycloalkyl; bicycloalkyl; aryl; acyl; benzoyl;

or NR⁷ and R⁸ taken together form a 4-12 membered mononitrogen containing monocyclic or bicyclic ring optionally substituted with one or more substituent selected from lower alkyl, carboxyl derivatives, aryl or hydroxy and wherein said ring optionally contains a heteroatom selected from the group consisting of O, N and S;

R⁵ is selected from the group consisting of H, cycloalkyl and alkyl;

or

A¹ is

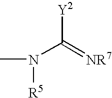

wherein Y² is selected from the group consisting of alkyl; cycloalkyl; bicycloalkyl; aryl; monocyclic heterocycles;

Z₁ is selected from the group consisting of CH₂, O, CH₂O, NRₖ, CO, S, SO, CH(OH) and SO₂, wherein Rₖ is selected from H or lower alkyl;

Z₂ is a 1-5 carbon linker optionally containing one or more heteroatom selected from the group consisting of O, S and N; alternatively Z₁-Z₂ may further contain a carboxamide, sulfone, sulfonamide, alkenyl, alkynyl, or acyl group;

wherein the carbon and nitrogen atoms of Z₁-Z₂ are optionally substituted by alkyl, cycloalkyl, alkoxy, thioalkyl, alkylsulfone, aryl, alkoxyalkyl, hydroxy, alkylamino, heteroaryl, alkenyl, alkynyl, carboxyalkyl, halogen, haloalkyl or acylamino;

n is an integer 0, 1 or 2;

R^{c} is selected from the group consisting of hydrogen; alkyl; cycloalkyl; halogen, hydroxy, nitro, alkoxy, amino, haloalkyl, aryl, heteroaryl, alkoxyalkyl, aminoalkyl, hydroxyalkyl, thioalkyl, alkylamino, arylamino, alkylsulfonylamino, acyl, acylamino, sulfonyl, sulfonamide, allyl, alkenyl, methylenedioxy, ethylenedioxy, alkynyl, alkynylalkyl, carboxy, alkoxycarbonyl, carboxamido, cyano, and —(CH₂)ₙCOR wherein n is 0-2 and R is selected from hydroxy, alkoxy, alkyl and amino;

X is selected from the group consisting of O, CO, SO₂, NR^{m} and (CHR^{p})ₙ; wherein R^{p} and R^{m} are H or alkyl;

R^{b} is X₃—R^{h} wherein X₃ is selected from the group consisting of O, S and NR^{j} wherein R^{h} and R^{j} are independently selected from the group consisting of H, alkyl, acyl, aryl, aralkyl and alkoxyalkyl; and

It is another object of the invention to provide pharmaceutical compositions comprising compounds of the Formula I. Such compounds and compositions are useful in selectively inhibiting or antagonizing the α_{V}β₃ and/or α_{V}β₅ integrin(s) and therefore in another embodiment the present invention relates to a method of selectively inhibiting or antagonizing the α_{V}β₃ and/or α_{V}β₅ integrin(s). The invention further involves treating or inhibiting pathological conditions associated therewith such as osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis, periodontal disease, psoriasis, smooth muscle cell migration including restenosis or atherosclerosis in a mammal in need of such treatment. Additionally, such pharmaceutical agents are useful as antiviral agents, antifungals and antimicrobials. The compounds of the present invention may be used alone or in combination with other pharmaceutical agents.

### DETAILED DESCRIPTION

The present invention relates to a class of compounds represented by the Formula I, described above.

In the embodiment of the present invention,

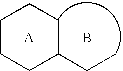

is selected from the group consisting of

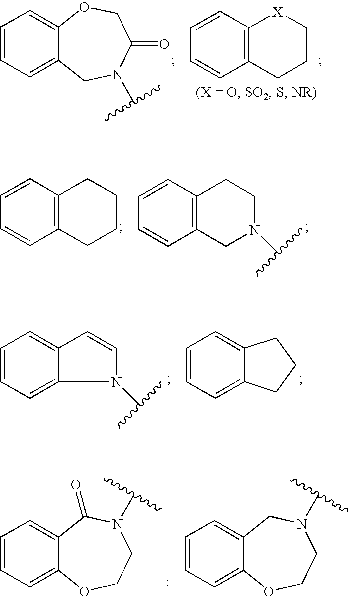

all optionally substituted and bonded to X and Z₁ at any position.

A¹ is a 5-9 membered monocyclic or 7-12 membered bicyclic heterocycle of the formula

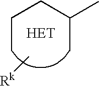

which includes the following heterocyclic ring systems containing at least one nitrogen atom:

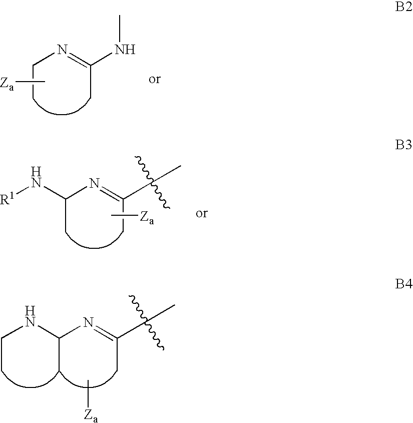

wherein Zₐ is H, alkyl, alkoxy, hydroxy, amine, alkylamine, dialkylamine, carboxyl, alkoxycarbonyl, hydroxyalkyl, halogen or haloalkyl and R¹ is H, alkyl, alkoxyalkyl, acyl, haloalkyl or alkoxycarbonyl. More specifically some examples include pyridylamino, imidazolylamino, morpholinopyridine, tetrahydronaphthyridine, oxazolylamino, thiazolylamino, pyrimidinylamino, quinoline, tetrahydroquinoline, imidazopyridine, benzimidazole, pyridone or quinolone.

The following heteroaryls include the ring systems described above.

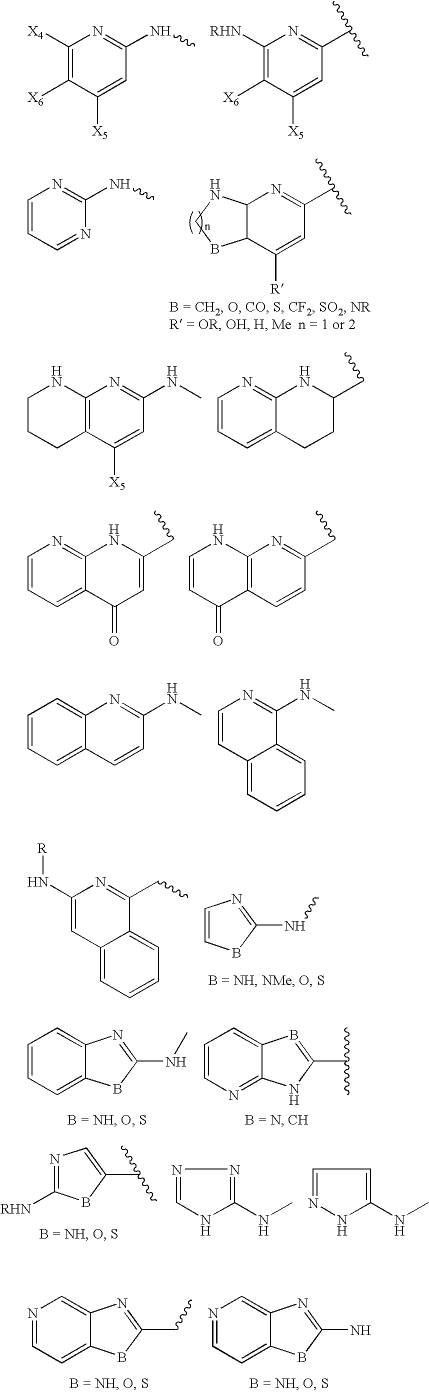

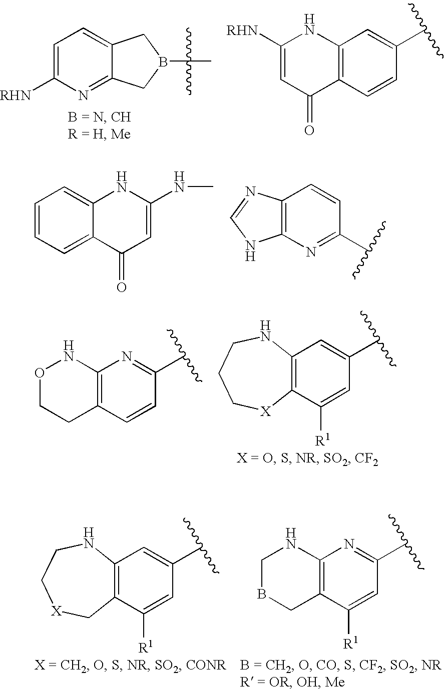

For the pyridyl derived heterocycle, the substituents X₄ and X₅ are selected from the group consisting of H, alkyl, branched alkyl, alkylamino, alkoxyalkylamino, haloalkyl, thioalkyl, halogen, amino, alkoxy, aryloxy, alkoxyalkyl, hydroxy, cyano or acylamino groups.

In another embodiment of the invention, the substituents X₄ and X₅ can be methyl, methoxy, amine, methylamine, trifluoromethyl, dimethylamine, hydroxy, chloro, bromo, fluoro and cyano. X₆ may preferentially be H, alkyl, hydroxy, halogen, alkoxy and haloalkyl. Alternately, the pyridyl ring can be fused with a 4-8 membered ring, optionally saturated or unsaturated. Some examples of these ring systems include tetrahydronaphthyridine, quinoline, tetrahydroquinoline, azaquinoline, morpholinopyridine, imidazopyridine and the like. The monocyclic ring systems such as imidazole, thiazole, oxazole, pyrazole, and the like, may contain an amino or alkylamino substituent at any position within the ring.

In another embodiment of the present invention, when Z₁ of Formula I is CO or SO₂, the linkage A¹-Z₂ of Formula I includes the heterocycle derived ring systems such as: pyridine, imidazole, thiazole, oxazole, benzimidazole, imidazopyridine and the like.

Other heterocycles for A¹-Z₂ of the present invention include

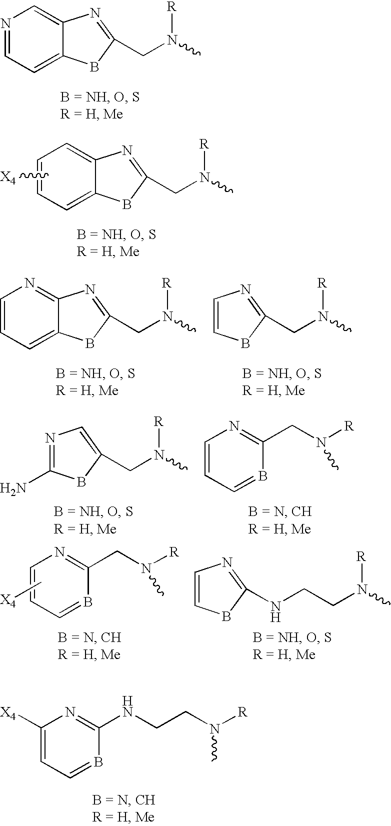

wherein X₄ is as defined above.

In another embodiment of the present invention, when the group

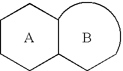

in Formula I is a tetrahydronaphthalene

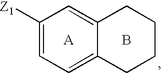

Z₁ may preferentially be S and A¹, Z₂, X and R^{b} as defined above. When Z₁ in tetrahydronaphthalene is a CH₂, the ring A¹ may be selected from the group consisting of:

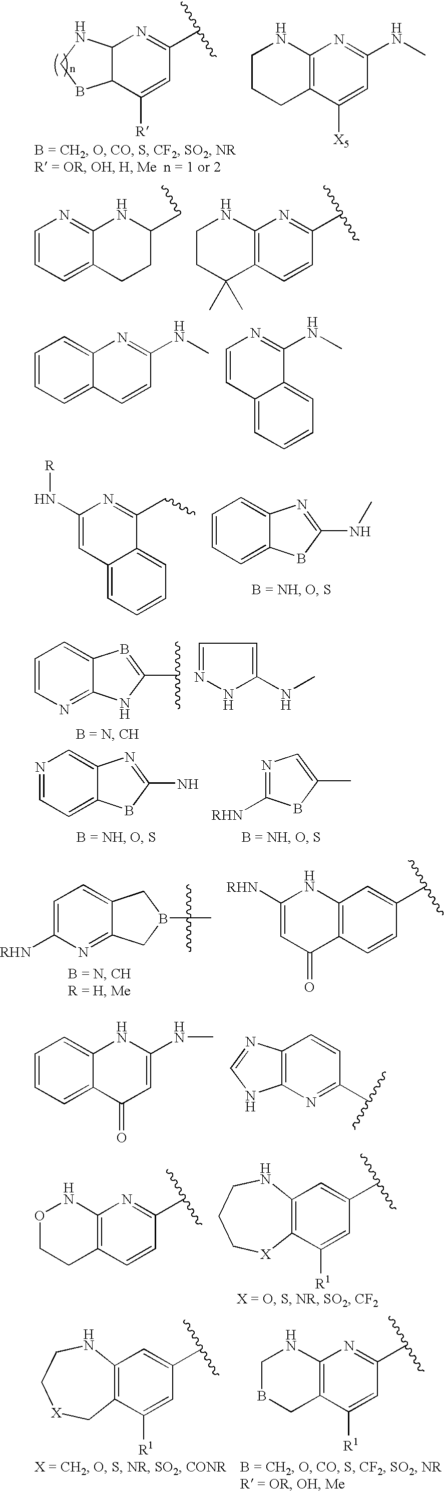

In another embodiment of the present invention, when the

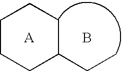

in Formula I is

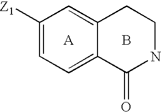

Z₁, Z₂, X and R^{b} may be as defined above. The ring A¹ may be selected from the group consisting of:

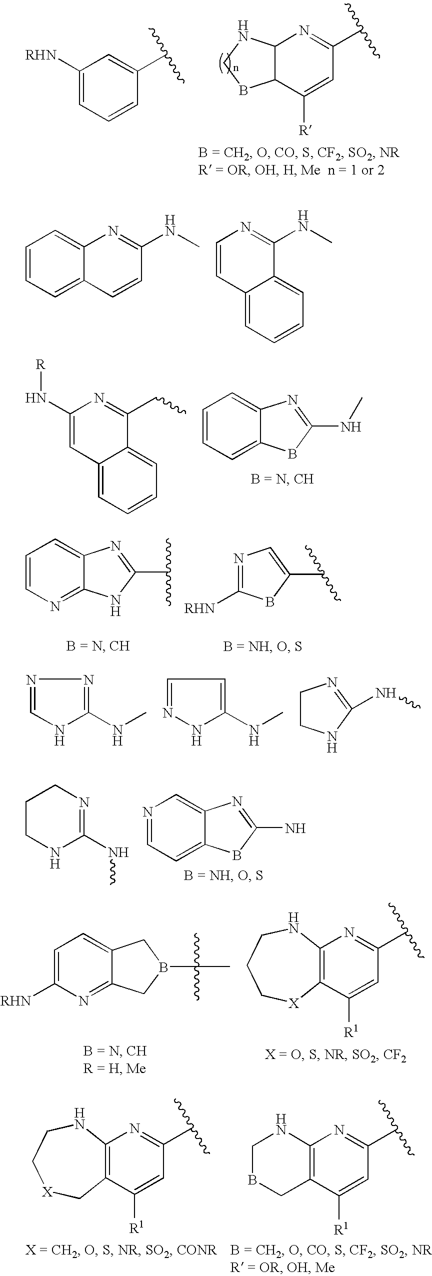

The invention further relates to pharmaceutical compositions containing therapeutically effective amounts of the compounds of Formula I.

The invention also relates to a method of selectively inhibiting or antagonizing the α_{V}β₃ integrin and/or the α_{V}β₅ integrin and more specifically relates to a method of inhibiting bone resorption, periodontal disease, osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis, smooth muscle cell migration, including restenosis and atherosclerosis by administering a therapeutically effective amount of a compound of the Formula I to achieve such inhibition together with a pharmaceutically acceptable carrier.

The following is a list of definitions of various terms used herein:

As used herein, the terms “alkyl” or “lower alkyl” refer to a straight chain or branched chain hydrocarbon radicals having from about 1 to about 10 carbon atoms, and more preferably 1 to about 6 carbon atoms. Examples of such alkyl radicals are methyl, ethyl, n-propyl, isopropyl, n-butyl, isobutyl, sec-butyl, t-butyl, pentyl, neopentyl, hexyl, isohexyl, and the like.

As used herein the terms “alkenyl” or “lower alkenyl” refer to unsaturated acyclic hydrocarbon radicals containing at least one double bond and 2 to about 6 carbon atoms, which carbon-carbon double bond may have either cis or trans geometry within the alkenyl moiety, relative to groups substituted on the double bond carbons. Examples of such groups are ethenyl, propenyl, butenyl, isobutenyl, pentenyl, hexenyl and the like.

As used herein the terms “alkynyl” or “lower alkynyl” refer to acyclic hydrocarbon radicals containing one or more triple bonds and 2 to about 6 carbon atoms. Examples of such groups are ethynyl, propynyl, butynyl, pentynyl, hexynyl and the like.

The term “cycloalkyl” as used herein means saturated or partially unsaturated cyclic carbon radicals containing 3 to about 8 carbon atoms and more preferably 4 to about 6 carbon atoms. Examples of such cycloalkyl radicals include cyclopropyl, cyclopropenyl, cyclobutyl, cyclopentyl, cyclohexyl, 2-cyclohexen-1-yl, and the like.

The term “aryl” as used herein denotes aromatic ring systems composed of one or more aromatic rings. Preferred aryl groups are those consisting of one, two or three aromatic rings. The term embraces aromatic radicals such as phenyl, pyridyl, naphthyl, thiophene, furan, biphenyl and the like.

As used herein, the term “cyano” is represented by a radical of the formula 1

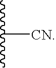

The terms “hydroxy” and “hydroxyl” as used herein are synonymous and are represented by a radical of the formula 2

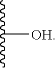

As used herein the term “alkoxy” refers to straight or branched chain oxy containing radicals of the formula —OR²⁰, wherein R²⁰ is an alkyl group as defined above. Examples of alkoxy groups encompassed include methoxy, ethoxy, n-propoxy, n-butoxy, isopropoxy, isobutoxy, sec-butoxy, t-butoxy and the like.

As used herein the terms “arylalkyl” or “aralkyl” refer to a radical of

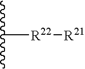

the formula 3 wherein R²¹ is aryl as defined above and R²² is an alkylene as defined above. Examples of aralkyl groups include benzyl, pyridylmethyl, naphthylpropyl, phenethyl and the like.

As used herein the term “nitro” is represented by a radical of the formula 4

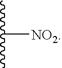

As used herein the term “halo” or “halogen” refers to bromo, chloro, fluoro or iodo.

As used herein the term “haloalkyl” refers to alkyl groups as defined above substituted with one or more of the same or different halo groups at one or more carbon atom. Examples of haloalkyl groups include trifluoromethyl, dichloroethyl, fluoropropyl and the like.

As used herein the term “carboxyl” or “carboxy” refers to a radical of the formula —COOH.

As used herein the term “carboxyl ester” refers to a radical of the formula —COOR²³ wherein R²³ is selected from the group consisting of H, alkyl, aralkyl or aryl as defined above.

As used herein the term “carboxyl derivative” refers to a radical of the formula

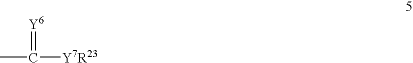

wherein Y⁶ and Y⁷ are independently selected from the group consisting of O, N or S and R²³ is selected from the group consisting of H, alkyl, aralkyl or aryl as defined above.

As used herein the term “amino” is represented by a radical of the formula —NH₂.

As used herein the term “alkylsulfonyl” or “alkylsulfone” refers to a radical of the

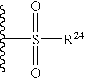

formula 6 wherein R²⁴ is alkyl as defined above.

As used herein the term “alkylthio” refers to a radical of the formula —SR²⁴ wherein R²⁴ is alkyl as defined above.

As used herein the term “sulfonic acid” refers to a radical of the

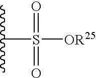

formula 7 wherein R²⁵ is alkyl as defined above.

As used herein the term “sulfonamide” or “sulfonamido” refers to a radical of the

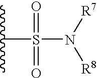

formula 8 wherein R⁷ and R⁸ are as defined above.

As used herein the term “fused aryl” refers to an aromatic ring such as the aryl groups defined above fused to one or more phenyl rings. Embraced by the term “fused aryl” is the radical naphthyl and the like.

As used herein the terms “monocyclic heterocycle” or “monocyclic heterocyclic” refer to a monocyclic ring containing from 4 to about 12 atoms, and more preferably from 5 to about 10 atoms, wherein 1 to 3 of the atoms are heteroatoms selected from the group consisting of oxygen, nitrogen and sulfur with the understanding that if two or more different heteroatoms are present at least one of the heteroatoms must be nitrogen. Representative of such monocyclic heterocycles are imidazole, furan, pyridine, oxazole, pyran, triazole, thiophene, pyrazole, thiazole, thiadiazole, and the like.

As used herein the term “fused monocyclic heterocycle” refers to a monocyclic heterocycle as defined above with a benzene fused thereto. Examples of such fused monocyclic heterocycles include benzofuran, benzopyran, benzodioxole, benzothiazole, benzothiophene, benzimidazole and the like.

As used herein the term “methylenedioxy” refers to the

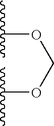

radical and the term “ethylenedioxy” refers to the radical

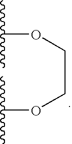

As used herein the term “4-12 membered dinitrogen containing Heterocycle” refers to a radical of the formula

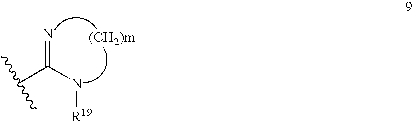

9 wherein m is an integer 1 to 7 and R¹⁹ is H, alkyl, aryl, or aralkyl and more preferably refers to 4-9 membered ring and includes rings such as imidazoline.

As used herein the term “5-membered optionally substituted heteroaromatic ring” includes for example a radical of the formula

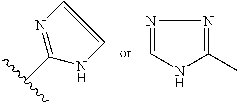

and “5-membered heteroaromatic ring fused with a phenyl” refers to such a “5-membered heteroaromatic ring” with a phenyl fused thereto. Representative of such 5-membered heteroaromatic rings fused with a phenyl is benzimidazole.

As used herein the term “bicycloalkyl” refers to a bicyclic hydrocarbon radical containing 6 to about 12 carbon atoms which is saturated or partially unsaturated.

As used herein the term “acyl” refers to a radical of the formula

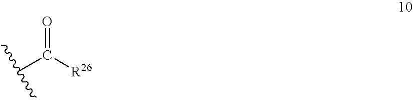

wherein R²⁶ is alkyl, alkenyl, alkynyl, aryl or aralkyl and optionally substituted thereon as defined above. Encompassed by such radical are the groups acetyl, benzoyl and the like.

As used herein the term “thio” refers to a radical of the formula

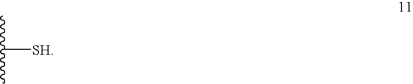

As used herein the term “sulfonyl” refers to a radical of the formula

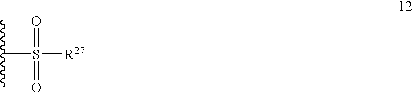

wherein R²⁷ is alkyl, aryl or aralkyl as defined above.

As used herein the term “haloalkylthio” refers to a radical of the formula —S—R²⁸ wherein R²⁸ is haloalkyl as defined above.

As used herein the term “aryloxy” refers to a radical of the formula

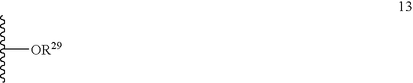

wherein R²⁹ is aryl as defined above.

As used herein the term “acylamino” refers to a radical of the formula

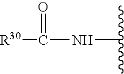

wherein R³⁰ is alkyl, aralkyl or aryl as defined above.

As used herein the term “amido” refers to a radical of the formula

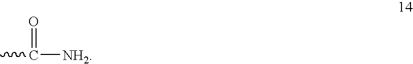

As used herein the term “alkylamino” refers to a radical of the formula —NHR³² wherein R³² is alkyl as defined above.

As used herein the term “dialkylamino” refers to a radical of the formula —NR³³R³⁴ wherein R³³ and R³⁴ are the same or different alkyl groups as defined above.

As used herein the term “trifluoromethyl” refers to a radical of the formula

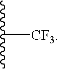

As used herein the term “trifluoroalkoxy” refers to a radical of the formula

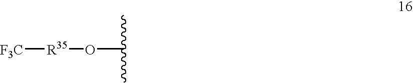

wherein R³⁵ is a bond or an alkylene as defined above.

As used herein the term “alkylaminosulfonyl” or “alkylsulfonamide” refer to a radical of the formula

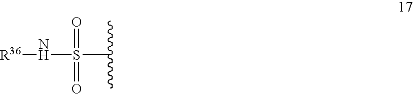

wherein R³⁶ is alkyl as defined above.

As used herein the term “alkylsulfonylamino” refers to a radical of the formula

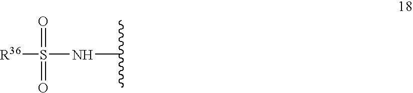

wherein R³⁶ is alkyl as defined above.

As used herein the term “trifluoromethylthio” refers to a radical of the formula

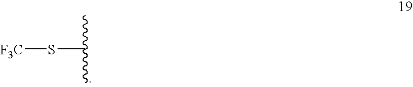

As used herein the term “trifluoromethylsulfonyl” refers to a radical of the formula

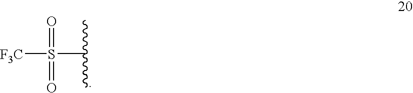

As used herein the term “4-12 membered mono-nitrogen containing monocyclic or bicyclic ring” refers to a saturated or partially unsaturated monocyclic or bicyclic ring of 4-12 atoms and more preferably a ring of 4-9 atoms wherein one atom is nitrogen. Such rings may optionally contain additional heteroatoms selected from nitrogen, oxygen or sulfur. Included within this group are morpholine, piperidine, piperazine, thiomorpholine, pyrrolidine, proline, azacycloheptene and the like.

As used herein the term “benzyl” refers to the radical

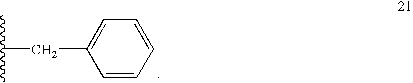

As used herein the term “phenethyl” refers to the radical

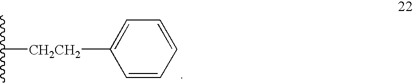

As used herein the term “4-12 membered mono-nitrogen containing monosulfur or monooxygen containing heterocyclic ring” refers to a ring consisting of 4 to 12 atoms and more preferably 4 to 9 atoms wherein at least one atom is a nitrogen and at least one atom is oxygen or sulfur. Encompassed within this definition are rings such as thiazoline and the like.

As used herein the term “alkylcarbonyl” refers to a radical of the formula

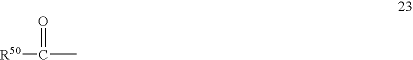

wherein R⁵⁰ is alkyl as defined above.

As used herein the term “arylcarbonyl” refers to a radical of the formula

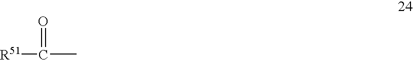

wherein R⁵¹ is aryl as defined above.

As used herein the term “alkoxycarbonyl” refers to a radical of the formula

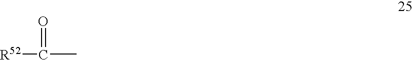

wherein R⁵² is alkoxy as defined above.

As used herein the term “aryloxycarbonyl” refers to a radical of the formula

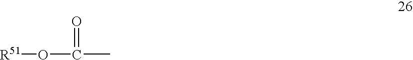

wherein R⁵¹ is aryl as defined above.

As used herein the term “haloalkylcarbonyl” refers to a radical of the formula

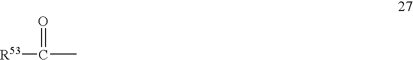

wherein R⁵³ is haloalkyl as defined above.

As used herein the term “haloalkoxycarbonyl” refers to a radical of the formula

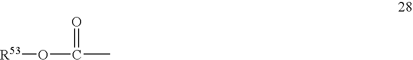

wherein R⁵³ is haloalkyl as defined above.

As used herein the term “alkylthiocarbonyl” refers to a radical of the formula

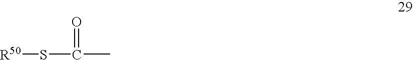

wherein R⁵⁰ is alkyl as defined above.

As used herein the term “arylthiocarbonyl” refers to a radical of the formula

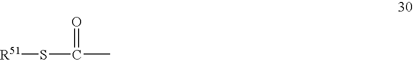

wherein R⁵¹ is aryl as defined above.

As used herein the term “acyloxymethoxycarbonyl” refers to a radical of the formula

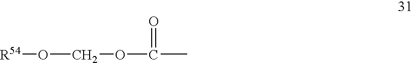

wherein R⁵⁴ is acyl as defined above.

As used herein the term “arylamino” refers to a radical of the formula R⁵¹—NH— wherein R⁵¹ is aryl as defined above.

As used herein the term “alkylamido” refers to a radical of the formula

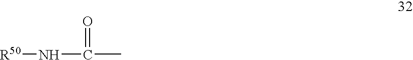

wherein R⁵⁰ is alkyl as defined above.

As used herein the term “N,N-dialkylamido” refers to a radical of the formula

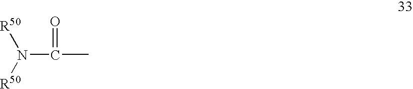

wherein R⁵⁰ is the same or different alkyl group as defined above.

As used herein the term “acyloxy” refers to a radical of the formula R⁵⁵—O— wherein R⁵⁵ is acyl as defined above.

As used herein the term “alkenylene” refers to a linear hydrocarbon radical of 1 to about 8 carbon atoms containing at least one double bond.

As used herein the term “alkoxyalkyl” refers to a radical of the formula

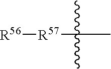

wherein R⁵⁶ is alkoxy as defined above and R⁵⁷ is alkylene as defined above.

As used herein the term “alkynylalkyl” refers to a radical of the formula R⁵⁹—R⁶⁰— wherein R⁵⁹ is alkynyl as defined as above and R⁶⁰ is alkylene as defined as above.

As used herein the term “alkynylene” refers to divalent alkynyl radicals of 1 to about 6 carbon atoms.

As used herein the term “allyl” refers of a radical of the formula —CH₂CH═CH₂.

As used herein the term “aminoalkyl” refers to a radical of the formula H₂N—R⁶¹ wherein R⁶¹ is alkylene as defined above.

As used herein the term “benzoyl” refers to the aryl radical C₆H₅—CO—.

As used herein the terms “carboxamide” or “carboxamido” refer to a radical of the formula —CO—NH₂.

As used herein the term “carboxyalkyl” refers to a radical HOOC—R⁶²— wherein R⁶² is alkylene as defined as above.

As used herein the term “carboxylic acid” refers to the radical —COOH.

As used herein the term “ether” refers to a radical of the formula R⁶³O— wherein R⁶³ is selected from the group consisting of alkyl, aryl and heteroaryl.

As used herein the term “haloalkylsulfonyl” refers to a radical of the formula

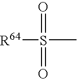

wherein the R⁶⁴ is haloalkyl as defined above.

As used herein the term “heteroaryl” refers to an aryl radical contain at least one heteroatom.

As used herein the term “hydroxyalkyl” refers to a radical of the formula HO—R⁶⁵— wherein R⁶⁵ is alkylene as defined above.

As used herein the term “keto” refers to a carbonyl group joined to 2 carbon atoms.

As used herein the term “lactone” refers to an anhydro cyclic ester produced by intramolecular condensation of a hydroxy acid with the elimination of water.

As used herein the term “olefin” refers to an unsaturated hydrocarbon radical of the type CₙH₂ₙ.

As used herein the term “sulfone” refers to a radical of the formula R⁶⁶—SO₂—.

As used herein the term “thioalkyl” refers to a radical of the formula R⁷⁷—S— wherein R⁷⁷ is alkyl as defined above.

As used herein the term “thioether” refers to a radical of the formula R⁷⁸—S— wherein R⁷⁸ is alkyl, aryl or heteroaryl.

As used herein the term “trifluoroalkyl” refers to an alkyl radical as defined above substituted with three halo radicals as defined above.

The term “composition” as used herein means a product which results from the mixing or combining of more than one element or ingredient.

The term “pharmaceutically acceptable carrier”, as used herein means a pharmaceutically acceptable material, composition or vehicle, such as a liquid or solid filler, diluent, excipient, solvent or encapsulating material, involved in carrying or transporting a chemical agent.

The term “therapeutically effective amount” shall mean that amount of drug or pharmaceutical agent that will elicit the biological or medical response of a tissue, system or animal that is being sought by a researcher or clinician.

The following is a list of abbreviations and the corresponding meanings as used interchangeably herein:

¹H-NMR=proton nuclear magnetic resonance

AcOH=acetic acid

Bn=benzyl

Boc=tert-butoxycarbonyl

Cat.=catalytic amount

CH₂Cl₂=dichloromethane

CH₃CN=acetonitrile

CHN analysis=carbon/hydrogen/nitrogen elemental analysis

DEAD=diethyl azodicarboxylate

DIBAL=diisobutylaluminum hydride

DI water=deionized water

DMF=N,N-dimethylformamide

DMSO=dimethylsulfoxide

Et=ethyl

EtI=ethyliodide

Et₂O=diethyl ether

Et₃N=triethylamine

EtOAc=ethyl acetate

EtOH=ethanol

g=gram(s)

h=hour(s)

HBTU=benzotriazol-1-yl-tetramethyluronium hexafluro phosphate

HPLC=high performance liquid chromatography

i-Pr=isopropyl

i-Prop=isopropyl

K₂CO₃=potassium carbonate

KOH=potassium hydroxide

L=Liter

LiOH=lithium hydroxide

Me=methyl

Mel=methyl iodide

MeOH=methanol

mg=milligram

MgSO₄=magnesium sulfate

min—minute(s)

ml=milliliter

mL=milliliter

MS=mass spectroscopy

MTBE=methyl t-butyl ether

N₂=nitrogen

NaH—sodium hydride

NaHCO₃=sodium bicarbonate

NaOH=sodium hydroxide

NaOMe=sodium methoxide

Na₂PO₄=sodium phosphate

Na₂SO₄=sodium sulfate

NH₄HCO₃=ammonium bicarbonate

NH₄⁺HCO₂⁻=ammonium formate

NH₄OH=ammonium hydroxide

NMR=nuclear magnetic resonance

PPh₃=triphenylphosphine

Pd=palladium

Pd/C=palladium on carbon

Ph=phenyl

Pt=platinum

Pt/C=platinum on carbon

RPHPLC=reverse phase high performance liquid chromatography

RT=room temperature

t-BOC=tert-butoxycarbonyl

TFA=trifluoroacetic acid

THF=tetrahydrofuran

Δ=heating the reaction mixture

The compounds as shown above can exist in various isomeric forms and all such isomeric forms are meant to be included. Tautomeric forms are also included as well as pharmaceutically acceptable salts of such isomers and tautomers.

In the structures and formulas herein, a bond drawn across a bond of a ring can be to any available atom on the ring.

The term “pharmaceutically acceptable salt” refers to a salt prepared by contacting a compound of Formula I with an acid whose anion is generally considered suitable for human consumption. Examples of pharmacologically acceptable salts include the hydrochloride, hydrobromide, hydroiodide, sulfate, phosphate, propionate, lactate, maleate, malate, succinate, tartrate salts and the like. Furthermore, where the compounds of the invention carry an acidic moiety, suitable pharmaceutically acceptable salts thereof may include alkali metal salts, e.g., sodium or potassium salts; or alkaline earth metal salts. All of the pharmacologically acceptable salts may be prepared by conventional means. (See Berge et al., J. Pharm. Sci., 66(1), 1-19 (1977) for additional examples of pharmaceutically acceptable salts.)

The present invention includes within its scope prodrugs of compounds of Formula I. These prodrugs are typically derivatives of the compounds of Formula I which are convertible to the active compounds on in-vivo exposure. These compounds may be derivatives of carboxylic acid (such as ester, amide, orthoester, urea and the like). Similarly derivatives of amine, hydroxy or other functional groups may be used as handles for pro-drug formation. Thus in the present invention, administering a compound for treatment of various conditions would include compounds specifically disclosed or the compounds which may not be specifically disclosed but would be converted to the specifically disclosed compound of Formula 1 on in-vivo administration. The methods described in literature (e.g., Design of pro-drugs, H. Bundgaard, Elsevier, 1985; Annual reports in Medicinal Chemistry, Vol 10, R. V. Heinzelman, ed.: Academic Press, 306-326, 1975) may be used for the preparation of prodrugs.

The compounds of the present invention may be chiral or achiral. These compounds may exist as racemic mixtures, diastereomers or pure enantiomers. For a chiral compound of present invention, separate enantiomers or all mixtures of diastereomers are included.

For the selective inhibition or antagonism of α_{V}β₃ and/or α_{V}β₅ integrins, compounds of the present invention may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, transmuscular infusion techniques or intraperitonally.

The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

Accordingly, the present invention provides a method of treating conditions mediated by selectively inhibiting or antagonizing the α_{V}β₃ and/or α_{V}β₅ cell surface receptor which method comprises administering a therapeutically effective amount of a compound selected from the class of compounds depicted in the above formulas, wherein one or more compound is administered in association with one or more non-toxic, pharmaceutically acceptable carriers and/or diluents and/or adjuvants (collectively referred to herein as “carrier” materials) and if desired other active ingredients. More specifically, the present invention provides a method for inhibition of the α_{V}β₃ and/or α_{V}β₅ cell surface receptors. Most preferably the present invention provides a method for inhibiting bone resorption, treating osteoporosis, inhibiting humoral hypercalcemia of malignancy, treating Paget's disease, inhibiting tumor metastasis, inhibiting neoplasia (solid tumor growth), inhibiting angiogenesis including tumor angiogenesis, treating retinopathy including macular degeneration and diabetic retinopathy, inhibiting arthritis, psoriasis and periodontal disease, and inhibiting smooth muscle cell migration including restenosis.

Based upon standard laboratory experimental techniques and procedures well known and appreciated by those skilled in the art, as well as comparisons with compounds of known usefulness, the compounds of Formula I can be used in the treatment of patients suffering from the above pathological conditions. One skilled in the art will recognize that selection of the most appropriate compound of the invention is within the ability of one with ordinary skill in the art and will depend on a variety of factors including assessment of results obtained in standard assay and animal models.

Treatment of a patient afflicted with one of the pathological conditions comprises administering to such a patient an amount of compound of the Formula I which is therapeutically effective in controlling the condition or in prolonging the survivability of the patient beyond that expected in the absence of such treatment. As used herein, the term “inhibition” of the condition refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

As stated previously, the compounds of the invention can be used in a variety of biological, prophylactic or therapeutic areas. It is contemplated that these compounds are useful in prevention or treatment of any disease state or condition wherein the α_{V}β₃ and/or α_{V}β₅ integrin plays a role.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 1.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regiment.

For administration to a mammal in need of such treatment, the compounds in a therapeutically effective amount are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulphuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The pharmaceutical compositions useful in the present invention may be subjected to conventional pharmaceutical operations such as sterilization and/or may contain conventional pharmaceutical adjuvants such as preservatives, stabilizers, wetting agents, emulsifiers, buffers, etc.

In another embodiment, the present invention provides treatment or prevention of a neoplasia disease in a mammal by combining one or more α_{V}β₃ integrin antagonists of the present invention with one or more chemotherapeutic agents. Among chemotherapeutic agents that may be used in combination with the α_{V}β₃ antagonist compounds include but are not limited to 5-fluorouacil, cyclophosphamide, cisplatin, taxol, and doxorubicin are preferred. Other chemotherapeutics useful in combination and within the scope of the present invention include but are not limited to buserelin, topoisomerase inhibitors such as topotecan and irinotecan, mitoxantrone, BCNU, CPT-11, chlorotranisene, chromic phosphate, gemcitabine, dexamethasone, estradiol, estradiol valerate, estrogens conjugated and esterified, estrone, ethinyl estradiol, floxuridine, goserelin, hydroxyurea, carboplatin, melphalan, methotrexate, mitomycin and prednisone.

The methods and combinations using one provide treatment or prevention of a neoplasia disease in a mammal using one or more αᵥβ₃ integrin antagonists described above with one or more chemotherapeutic agents described above. The method comprises treating a mammal with a therapeutically effective amount of an αᵥβ₃ integrin antagonist in combination with a chemotherapeutic agent.

There are five major classes of chemotherapeutic agents currently in use for the treatment of cancer: natural products and their derivatives; anthracyclins; alkylating agents; antimetabolites; and hormonal agents. Chemotherapeutic agents are often referred to as antineoplastic agents. The alkylating agents are believed to act by alkylating and cross-linking guanine and possibly other bases.

In DNA, arresting cell division. Typical alkylating agents include nitrogen mustards, ethyleneimine compounds, alkyl sulfates, cislatin, and various nitrosoureas. A disadvantage with these compounds is that they not only attack malignant cells, but also other cells which are naturally dividing, such as those of bone marrow, skin, gastrointestinal mucosa and fetal tissue.

Antimetaloties are typically reversible or irreversible enzyme inhibitors, or compounds that otherwise interfere with the replication, translation or transcription of nucleic acids.

Several synthetic nucleosides have been identified that exhibit anticancer activity. A well-known nucleoside derivative with strong anticancer activity is 5-fluorouacil. 5-fluorouacil has been used clinically in the treatment of malignant tumors, including, for example, carcinomas, sarcomas, skin cancer, cancer of the digestive organs, and breast cancer. 5-fluoroucil, however, causes serious adverse reactions such as nausea, alopecia, stomatites, leukocytic thrombocytopenia, anorexia, pigmentation and edema.

Cytosine arabinoside (also referred to as Cytarabin, araC, and Cytosar) is a nucleoside analog of deoxycytidine that was first synthesized in 1950 and introduced into clinical medicine in 1963. It is currently an important drug in the treatment of acute myeloid leukemia. It is also active against acute lymphocytic leukemia, and to a lesser extent, is useful in chronic myelocytic leukemia and non-Hodgkin's lymphoma.

The following table (Table 1) provides illustrative examples of median dosages for selected cancer agents that may be used in combination with a αᵥβ₃ integrin antagonist agent. It should be noted that the specific dose regimen for the chemotherapeutic agents below will depend upon dosing considerations based upon a variety of factors including the type of neoplasia; the state of the neoplasm, the age, weight, sex and medical condition of the patient; the route of administration, the renal and hepatic function of the patient; and the particular combination employed.

TABLE 1
NAME OF CHEMOTHERAPEUTIC AGENT

| | | |
|---|---|---|
| Asparaginase | 10,000 | units |
| Bleomycin Sulfate | 15 | Units |
| Carboplatin | 50-450 | mg |
| Carmustine | 100 | mg |
| Cisplatin | 10-50 | mg |
| Cladribine | 10 | mg |
| Cyclophosphmide | 100 | mg.-2 gm. |
| (lyophilized) | | |
| Cyclophosphamide | 100 | mg.-2 gm. |
| (non-lyophilized) | | |
| Cytarabine | 100 | mg.-2 gm. |
| (lyophilized powder) | | |
| Dacarbazine | 100 | mg.-200 mg. |
| Dactinomycin | 0.5 | mg. |
| Daunorubicin | 20 | mg. |
| Diethylstilbestrol | 250 | mg |
| Doxorubin | 10-150 | mg. |
| Etidronate | 300 | mg. |
| Etoposide | 100 | mg. |
| Floxuridine | 500 | mg. |
| Fludarabine Phosphate | 50 | mg. |
| Fluorouracil | 500 | mg.-5 gm. |
| Goserelin | 3.6 | mg. |
| Granisetron Hydrochloride | 1 | mg |
| Idarubicin | 5-10 | mg. |
| Ifosfamide | 1-3 | gm |
| Leucovorin Calcium | 50-350 | mg. |
| Leuprolide | 3.75-7.5 | mg. |
| Mechlorethamine | 10 | mg. |
| Medroxyprogeserone | 1 | gm. |
| Melphalan | 50 | gm. |
| Methotrexate | 20 | mg.-1 gm. |
| Mitomycin | 5-40 | mg. |
| Mitoxantrone | 20-30 | mg. |
| Ondansetron Hydrochloride | 40 | mg. |
| Paclitaxel | 30 | mg. |
| Pamidronate Disodium | 30-90 | mg. |
| Pegaspargase | 750 | units |
| Pilcamyican | 2,500 | mcgm. |
| Streptozocin | 1 | gm. |
| Thiotepa | 15 | mg. |
| Teniposide | 50 | mg. |
| Vinblastine | 10 | mg. |
| Vincristine | 1-5 | mg. |

The general synthetic sequences for preparing the compounds useful in the present invention are outlined in SCHEMES 1-5. Both an explanation of, and the actual procedures for, the various aspects of the present invention are described where appropriate. The following Schemes and Examples are intended to be merely illustrative of the present invention, and not limiting thereof in either scope or spirit. Those with skill in the art will readily understand that known variations of the conditions and processes described in the Schemes and Examples can be used to synthesize the compounds of the present invention.

Unless otherwise indicated all starting materials and equipment employed were commercially available.

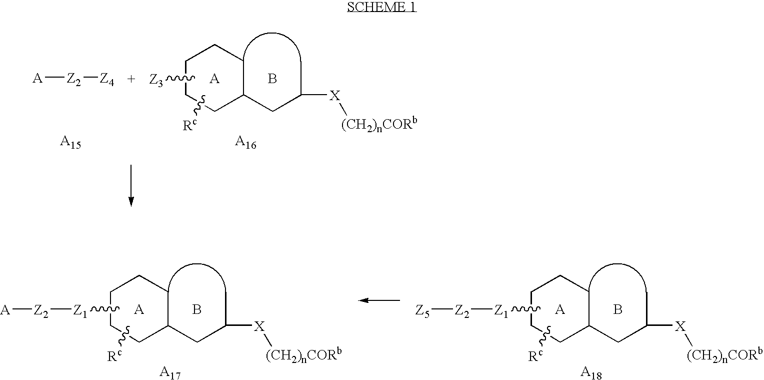

### SCHEME 1

The compounds of formula A₁₇ are generally prepared by reacting an intermediate of formula A₁₆ with a compound of the formula A₁₅. For example, when Z₃ is OH, SH or NHR, A₁₇ may be alkylated with A₁₅ (Z₄=Br or OMs) using base such as sodium hydride or potassium hydride preferably in a solvent such as dimethylsulfoxide or DMF. These reactions may preferentially be carried at 0° C. to approximately 40° C. Alternately, when Z₃ and Z₄ are both OH, the ether formation to product A₁₇ may be accomplished by using Mitsunobu reaction. This reaction may preferentially be carried out using a triarylphosphine (such as triphenylphoshine) and azodicarboxylate (such as diethyl azodicarboxylate, di-tert-butyl azodicarboxylate, di-iso-propyl azodicarboxylate) in solvents such as DMF, methylene chloride, THF and the like. When Z₃ carries a carboxylic acid or a sulfonic acid and Z₄ is an amine, the standard coupling conditions may be used to synthesize the carboxamide (CONH) or the sulfonamide (SO₂NH) containing targets A₁₇.

Alternately, the compounds of formula A₁₇ may be prepared by starting with compounds of general formula A₁₈. For example, when Z₅ in A₁₈ is NH₂, cyclic or acyclic guanidino containing compounds of formula A₁₇ may be synthesized by adopting the methodologies discussed in e.g., U.S. Pat. No. 5,852,210, U.S. Pat. No. 5,773,646. Similarly, compounds of formula A₁₈ (Z₅=NH₂) may be treated with appropriately substituted heteroaromatic system (such as 2-halopyridine N-oxide) to give the target compounds A₁₇. This reaction may preferentially be carried out by refluxing the intermediate A₁₈ and 2-halopyridine (such as 2-fluoropyridine, 2-chloropyridine N-oxide) in solvents such as tert-butyl alcohol, tert-amyl alcohol in the presence of base (such as sodium bicarbonate, sodium carbonate, potassium carbonate, potassium bicarbonate).

When compounds of formula A₁₇ contain N-oxide (e.g., pyridine N-oxide), the deoxygenation is preferentially carried out using transfer hydrogenation conditions (such as cyclohexene/Pd on carbon or ammonium formate and Pd on carbon). When R^{b} is OR, the hydrolysis of the resulting ester may be carried out using aqueous base such as sodium hydroxide, lithium hydroxide, potassium hydroxide using co-solvents such as methanol, ethanol or THF.

Compounds of the general formula A₁₅, A₁₆, A₁₈ may be prepared by methodologies discussed in SCHEMES below.

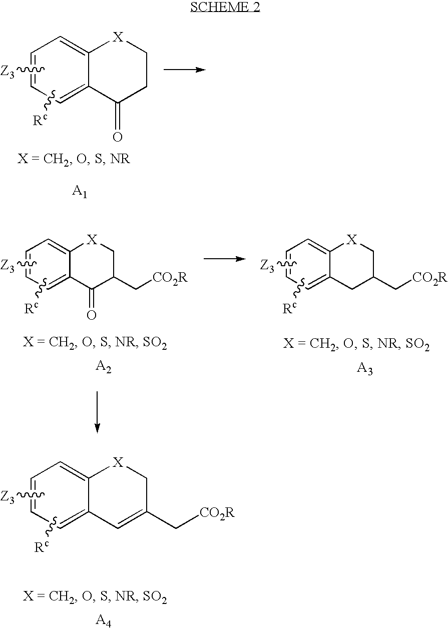

### SCHEME 2

Compounds of the formula A₂-A₄ may be prepared by starting with substituted 1-tetralones of formula A₁. Base catalyzed carbanion generation from A₁ followed by treatment with ethyl bromoacetate gives the intermediate A₂. Suitable bases for this reaction are lithium hexamethylsilazide, potassium hexamethylsilazide, lithium bis(trimethyl)silylamide, potassium (trimethylsilyl)amide, potassium hydride, potassium tert-butoxide and the like. Reduction of A₂ with sodium borohydride followed by dehydration using acid catalyst (such as p-toluene sulfonic acid) gives the intermediate A₄. Similarly hydrogenation of A₂ gives the intermediate A₃. This reaction may preferentially be carried out using Pd/C or Pt/C as catalyst and preferably in the presence of small amount of acid (such as phosphoric acid, acetic acid, hydrochloric acid). Intermediates of formula A₂-A₄ with X═SO₂ may be prepared from the corresponding mercapto intermediates (X═S) by oxidation with e.g; oxone, m-chloroperoxybenzoic acid and the like.

The intermediates A₂-A₄ are processed to the target compounds of Formula I by synthetic transformations outlined in SCHEME 1

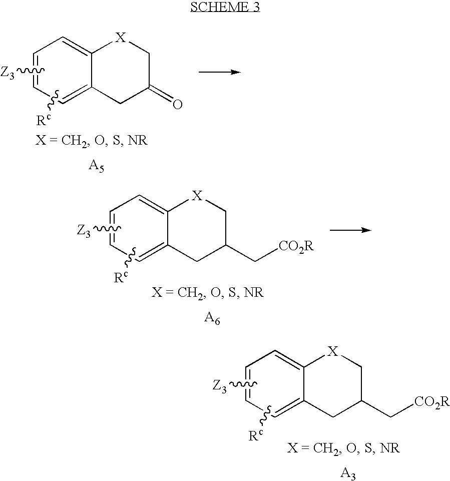

### SCHEME 3

Alternately, intermediates of formula A₃ may be prepared from 2-tetralone derived compounds A₅. Using Wittig or Horner-Emmons reaction, the compound A₅ is converted to the olefin containing intermediate A₆. This reaction is carried out using trialkyl phosphonoacetate (such as triethyl phosphonoacetate, trimethyl phosphonoacetate) and base (e.g., sodium hydride, sodium methoxide, sodium ethoxide). This reaction is generally done at low temperature (0-30° C.) and using solvent such as THF or DMF as solvents. The isomeric mixtures of olefin containing compounds are hydrogenated using e.g, Pd on carbon or Pt on carbon as catalyst. This reduction is carried under pressure of hydrogen (preferably 5-60 psi) to give the desired intermediate A₃. The intermediate A₃ is processed to the target compounds of Formula I by synthetic transformations outlined in SCHEME 1.

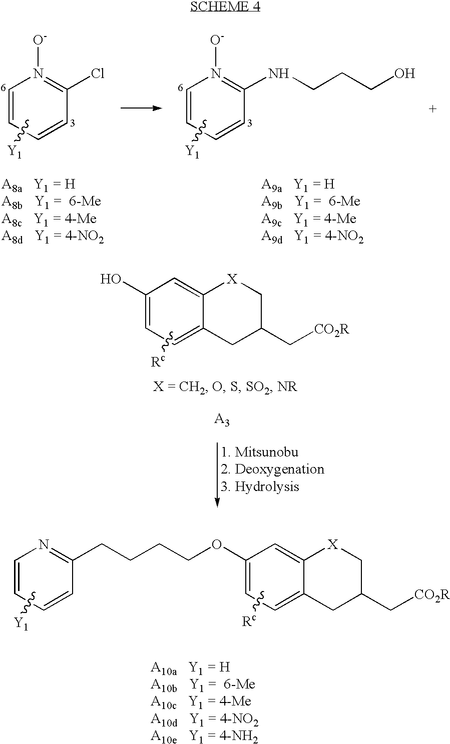

### SCHEME 4

The compounds of Formula I, wherein A is substituted pyridyl may be prepared by adopting the general synthetic SCHEME 4. For example, reaction of substituted 2-halopyridine N-oxide (such as A₈ₐ-A_{8d}) with e.g. 3-aminopropanol gives the intermediates A₉ₐ-A_{9d}. This reaction may preferentially be carried out by refluxing the intermediate 2-halopyridine N-oxide (such as 2-chloropyridine N-oxide) in solvents such as tert-butyl alcohol, tert-amyl alcohol in the presence of base (such as sodium bicarbonate, sodium carbonate, potassium carbonate, potassium bicarbonate). The preparative conditions described in WO 99/15508 (PCT US 98/19466) may be used for this transformation. Coupling of the intermediates A₉ₐ-A_{9d} with A₃ using Mitsunobu reaction gives the compounds containing the ether link. This reaction may preferentially be carried out using triarylphosphine (such as triphenylphoshine) and azodicarboxylate (such as diethyl azodicarboxylate, di-tert-butyl azodicarboxylate, di-iso-propyl azodicarboxylate) in solvents such as DMF, methylene chloride, or THF. N-Deoxygenation of resulting intermediates followed by hydrolysis of the ester gives the target compounds (A₁₀ₐ-A_{10d}). Reduction of the N-oxide bond may be accomplished using e.g., transfer hydrogenation (cyclohexene and Pd on carbon) or ammonium formate and Pd on carbon. The nitro group in 10_{d} may be hydrogenated using Pd on carbon or Pt on carbon as catalysts to give intermediate A₁₀ₑ. This transformation may be carried out using solvents such as methanol, ethanol or THF. The hydrolysis of the ester group may be carried using aqueous base (such as sodium hydroxide, lithium hydroxide or potassium hydroxide) in solvents such as methanol, ethanol and THF.

Compounds of Formula I containing heterocycle other than pyridyl can also be prepared using the scheme discussed above. For example reacting 2-bromopyrimidine or 1-chloroisoquinoline N-oxide with 3-aminopropanol gives the analogous intermediates as obtained in Step 1. The resulting intermediates could be elaborated as in SCHEME 4 to similarly give the pyrimidine, quinoline and isoquinoline containing target compounds.

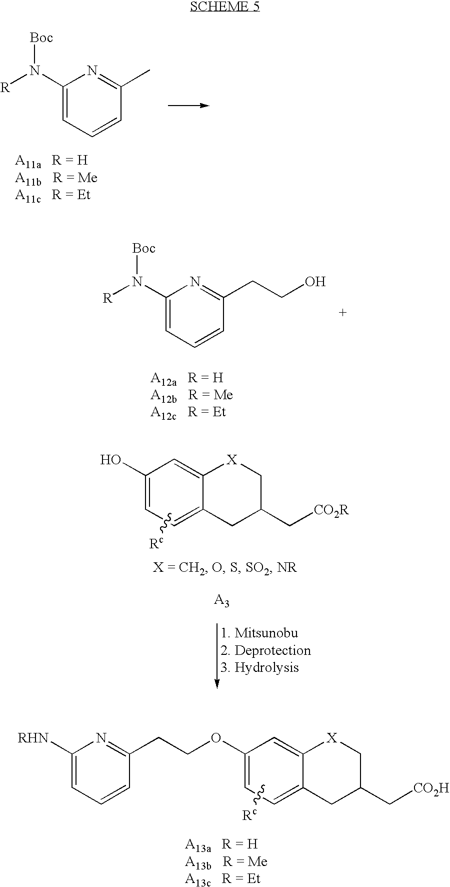

### SCHEME 5

Compounds of Formula I containing 6-amino substituents may be prepared as shown in SCHEME 5. The intermediate A_{12b} can be prepared as described in J. Med. Chem 43, 22, 2000. Boc-protected 2-amino-6-picoline (A₁₁ₐ) or its ethylated derivative (A_{11c}) are elaborated to A₁₂ₐ and A_{12c} as shown for case A_{12b} in the above publication. The ethylated intermediate A_{11c} may be prepared from Alia by alkylation using e.g.; Etl and base such as potassium carbonate, cesium carbonate. This reaction may preferentially be carried out in polar solvents such as dimethylformamide, dimethylacetamide. Mitsunobu reaction of A₁₂ₐ-A_{l2c}with A₃, gives the compounds containing the phenol ether. This reaction may be carried out using the conditions discussed in SCHEME 4. Removal of the Boc group using e.g., trifluoroacetic acid in solvents such as dichloromethane, followed by hydrolysis of the ester group as discussed in SCHEME 4 above gives the target compounds (A₁₃ₐ-A_{13c}).

### EXAMPLE 1

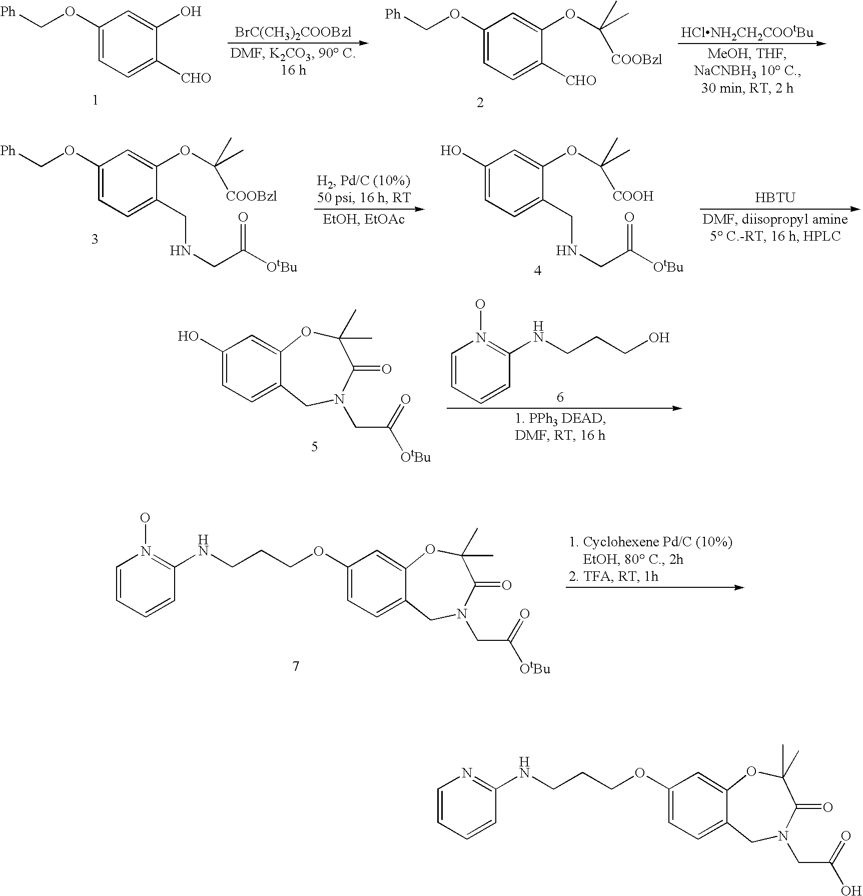

STEP 1

A mixture of 4-benzyloxy-2-hydroxybenzaldehyde (1) (10.0 g, 0.043 mol), 2-bromoisobutyrylbenzoate (16.5 g, 0.064 mol), potassium carbonate (6.7 g, 0.048 mol) in DMF (50.0 mL) was heated at 90° C. under argon for 16 hours. The resulting dark colored solution was cooled, diluted with cold water (100 mL), and extracted with EtOAc (3×50 mL). The combined organic extracts were washed with water (3×25 mL), dried (Na₂SO₄), and concentrated to dryness under reduced pressure. The resulting residue was purified by silica gel flash chromatography using 5% EtOAc in hexane as the eluent to afford 2-[2-formyl-5-(phenylmethoxy)phenoxy]-2-methylpropanoic acid, phenylmethyl ester (12.5 g) as a pale yellow liquid: ¹H-NMR (CDCl₃, 400 MHz) δ 10.31 (s, 1H), 7.78 (d, 1H, J=8.8 Hz), 7.36 (m, 10H) 6.62 (m, 1H), 6.17 (d, 1H, J=2.4 Hz), 5.15 (s, 2H), 4.83 (s, 2H), and 1.63 (s, 6H); ES-MS m/z 405 (MH⁺), HR-MS (ES) m/z calcd for C₂₅H₂₅O₅ (MH⁺) 405.1702, found 405.1705.

STEP 2

To a solution of t-butylglycinate hydrochloride (1.9 g, 0.011 mol) in MeOH (15 mL) at 10° C., was added NaCNBH₃ (0.7 g, 0.011 mol), followed by the addition of a solution of product from STEP 1 (3.7 g, 0.0091 mol) in MeOH (15 mL) and THF (10 mL). The reaction mixture was stirred at 10° C. for 30 minutes and at room temperature for 2 hours under anhydrous conditions. It was then quenched by the addition of glacial acetic acid (2 mL), and concentrated to dryness under reduced pressure. The resulting residue was partitioned between water (30 mL) and EtOAc (30 mL). The organic phase was washed water (3×25 mL), dried (Na₂SO₄), and concentrated to dryness under reduced pressure to give a yellow viscous liquid. This material was purified by silica gel flash chromatography using 75% EtOAc in hexane as the eluent to afford 2-[2-[[[2-(1,1-dimethylethoxy)-2-oxyethyl]-amino]methyl-5-hydroxyphenoxy]-2-methylpropanoic acid, phenylmethyl ester (3.8 g) as a colorless viscous liquid: ¹H-NMR (CDCl₃, 400 MHz) δ 7.27 (m, 10H), 7.21 (m, 1H) 6.60 (m, 1H), 6.38 (s, 1H), 5.14 (s, 2H), 4.91 (s, 2H), 4.08 (s, 2H), 3.52 (s, 2H), 1.68 (s, 6H), 1.45 (s, 9H); ES-MS m/z 520 (MH⁺), HR-MS (ES) m/z calcd for C₃₁H₃₈NO₅ (MH⁺) 520.2699, found 520.2691.

STEP 3

A solution of the product from STEP 2 (3.7 g, 0.071 mol) in EtOH (20 mL) and EtOAc (10.0 mL) was hydrogenated at 50 psi in the presence of Pd/C (10%, 2.7 g) for 16 hours at room temperature. The catalyst was removed by filtration, and the filtrate was concentrated to dryness to give 2-[2-[[[2-(1,1-dimethylethoxy)-2-oxoethyl]amino]methyl]-5-hydroxyphenoxy]-2-methylpropanoic acid (1.5 g) as a white amorphous powder: ¹H-NMR (CD₃OD) δ 7.14 (d, 1H, J=8.8 Hz), 6.44 (dd, 1H, J=2.4 Hz), 6.38 (d, 1H, J=2.4 Hz), 4.13 (s, 2H), 3.80 (s, 2H), 1.67 (s, 6H), 1.51 (s, 9H); ES-MS m/z 340 (MH⁺), HR-MS (ES) m/z calcd for C₁₇H₂₆NO₆(MH⁺) 340.1760, found 340.1757.

STEP 4

To a cold (5° C.) solution of the product from STEP 3 (0.9 g, 0.0265 mol) in DMF (10.0 mL) was added diisopropylethyl amine (0.5 mL), followed by the addition of 2-(H-benzotriazole-1-yl)-1,1,3,3-tetramethyluronium hexafluorophosphate (HBTU 1.1 g, 0.0029 mol), and the mixture was stirred at room temperature. After 1 hour, additional diisopropylethyl amine 90.5 mL) was added and stirring was continued for another 16 hours. DMF was distilled in vacuo and the residue was purified by reverse-phase HPLC using 10-90% CH₃CN/Water gradient (30 minutes) at a flow rate of 70 mL/min. Appropriate fractions were pooled and lyophylized to afford 2,3,4,5-tetrahydro-8-hydroxy-2,2-dimethyl-3-oxo-1,4-benzoxapine-4-acetic acid, 1,1-dimethylethyl ester (0.38 g): ¹H-NMR (CD₃OD) δ 7.18 (d, 1H, J=8.0 Hz), 6.61 (m, 1H,), 6.56 (br s, 1H), 4.50 (s, 2H), 4.12 (s, 2H), 1.60 (s, 6H), 1.48 (s, 9H); ES-MS m/z 322 (MH⁺), HR-MS (ES) m/z calcd for C₁₇H₂₄NO₅(MH⁺)322.1654, found 322.1643.

STEP 5

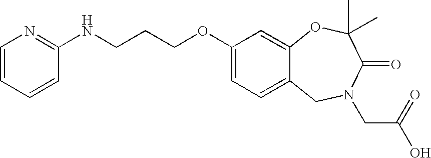

[2,2-dimethyl-3-oxo-8-[3-(pyridin-2-ylamino)propoxy]-2,3-dihydro-1,4-benzoxazepin-4(5H)-yl]acetic Acid

To a cold (5° C.) solution of the product from STEP 4 (0.6 g, 0.0019 mol) in DMF (10 mL), was added triphenyl phosphine (0.8 g, 0.0029 mol), followed by the addition of diethylazodicarboxylate (0.5 mL). After 5 minutes, 3-[1-oxido-2-pyridinyl) amino]-1-propanol (0.52 g, 0.0031 mol) was added and resulting mixture was stirred at room temperature for 16 hours. DMF was distilled in vacuo, and the residue was purified reverse-phase HPLC using 10-90% CH₃CN/Water gradient (30 minutes) at a flow rate of 70 mL/minute. Appropriate fractions were pooled and lyophylized to afford the desired product (0.25 g): HR-MS (ES) m/z calcd for C₂₅H₃₄N₃O₆(MH⁺) 472.2448, found 472.2442. This material was dissolved in EtOH (10.0 ml), cyclohexene (0.3 mL) and Pd/C (10%, 0.25 g) were added, and the mixture was heated to reflux for 2 hours. The reaction mixture was cooled, filtered, and the filtrate was concentrated to dryness under reduced pressure. The residue was then stirred with trifluoroacetic acid (1.0 mL) for 1 hour, concentrated to dryness, and the product was purified by reverse-phase HPLC using 10-90% CH₃CN/Water gradient (30 min) at a flow rate of 70 mL/minute. Appropriate fractions were pooled and lyophylized to afford [2,2-dimethyl-3-oxo-8-[3-(pyridin-2-ylamino)propoxy]-2,3-dihydro-1,4-benzoxazepin-4(5H)-yl]acetic acid, mono(trifluoroacetate) (0.11 g): ¹H-NMR (CD₃OD) δ 7.88 (m 1H), 7.77 (d, 1H, J=6.0 Hz), 7.21 (d, 1H, J=8.4 Hz), 7.03 (d, 1H, J=8.4 Hz), 6.85 (m, 1H), 6.86 (dd, 1H, J=2.4 Hz), 6.64 (d, 1H, J=2.4 Hz), 4.47 (s, 2H), 4.12 (s, 2H), 4.10 (t, 2H, J=6.0 Hz), 3.55 (t, 2H, J=6.0 Hz), 2.16 (m, 2H), 1.52 (s, 6H); ES-MS m/z 400 (MH⁺), HR-MS (ES) m/z calcd. for C₂₁H₂₆N₃O₅(MH⁺) 400.1867, found 400.1860.

### EXAMPLE 2

1,2,3,4-tetrahydro-6-[3-(2-pyridinylamino)propoxy]-2-naphthaleneacetic Acid

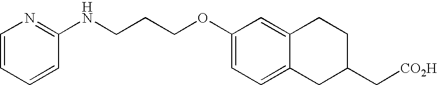

STEP 1

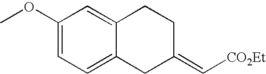

To a stirred suspension of 50% NaH (1.1 g, 22.8 mmole), in THF (50 ml) was added triethyl phosphonoacetate (4.48 g, 20 mmole) in THF (10 ml) drop wise over 5 minutes under nitrogen atmosphere. The mixture was stirred at room temperature for 30 minutes. 6-Methoxy-2-tetralone (3.52 g, 20 mmole) in THF (10 ml) was added, and the mixture was stirred under nitrogen atmosphere at ambient temperature for 2 hours. The mixture was quenched with water and was extracted with 3×50 ml portions of ethyl acetate. The organic extracts were washed with water, dried over Na₂SO₄ and concentrated in vacuo to give the above compound (2.89 g) as oily gum.

¹H NMR was consistent with the proposed structure.

STEP 2

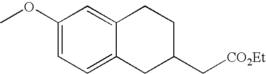

A solution of the product from STEP 1 (2.05 g, 8.7 mmole) in ethanol was shaken in a Parr hydrogenation apparatus with 5% Pd on carbon under 60 psi hydrogen pressure at ambient temperature for 2 hours. The mixture was filtered and concentrated in vacuo to give the product (1.95 g) as a thick liquid. ¹H NMR was consistent with the proposed structure.

STEP 3

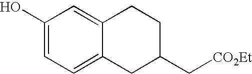

To a stirred solution of the product of STEP 2 (1.95 g, 7.93 mmole) in methylene chloride (15 ml) at −10° C., boron tribromide (15 ml, 1M solution in methylene chloride) was added. The mixture was stirred at 0° C. for 20 minutes under nitrogen atmosphere. The reaction mixture was quenched with ethanol (4 ml) and was concentrated in vacuo leaving behind an oily residue which was dissolved in ethyl acetate and was washed with water and brine. The organic extract was dried over Na₂SO₄ and concentrated in vacuo to give an oily residue. The residue was chromatographed (silica gel, hexane/ethyl acetate 7/3) to yield the desired product (1.41 g) as oily gum. ¹H NMR was consistent with the proposed structure.

STEP 4

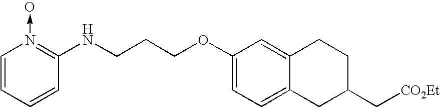

To a stirred solution of product of STEP 3 (1.4 g) and 2-[3-hydroxy-1-propyl)-amino]pyridine-N-oxide (1.09 g) and triphenylphosphine (1.7 g) in DMF (10 ml) at 0° C., a solution of diisopropylazodicarboxylate (1.25 ml) was added drop wise. The reaction mixture was stirred at ambient temperature for 18 hours. The mixture was diluted with water and was extracted with ethyl acetate. The organic extract was washed with water and dried over Na₂SO₄ and concentrated to give an oily residue. The residue was chromatographed (silica gel, methylene chloride/methanol/ammonium hydroxide 94/5/1) to give the desired product (681 mg) as a clear viscous oil. ¹H NMR was consistent with the proposed structure.

STEP 5

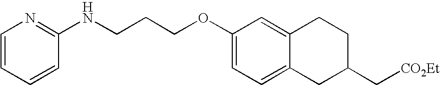

A solution of the product of STEP 4 (400 mg), 10% Pd on carbon (300 mg) and cyclohexene (3 ml) in isopropanol (12 ml) was refluxed under nitrogen atmosphere for 2.5 hours. The mixture was cooled to room temperature, filtered through a celite pad and washed with ethyl acetate. The filtrate was concentrated to give the desired product (288 mg) as clear viscous oil. ¹H NMR was consistent with the proposed structure.

STEP 6

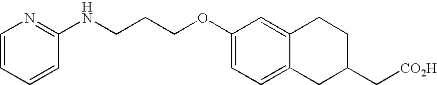

1,2,3,4-tetrahydro-6-[3-(2-pyridinylamino)propoxy]-2-naphthaleneacetic Acid

A solution of the product of STEP 5 (250 mg) in methanol (2.5 ml) and THF (2.5 ml) and 1N NaOH (2.5 ml) was stirred at room temperature for 1 hour. The reaction mixture was quenched with TFA (2 ml) and concentrated. The residue was chromatographed on reverse phase HPLC using acetonitrile/water (0.5% TFA) gradient to give the desired product (130 mg) as a viscous oil. ¹H NMR was consistent with the proposed structure. Anal Calcd for C₂₀H₂₄N₂O₃₀.1.1 TFA: C, 57.24; H, 5.43; N, 6.01; Found: C, 57.46; H, 5.50; N, 6.21.

### EXAMPLE 3

1,2,3,4-tetrahydro-6-[3-(2-pyridinylamino)propoxy]-2-isoquinolineacetic Acid

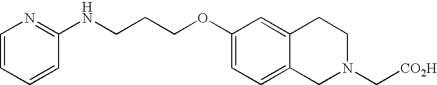

STEP 1

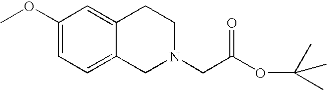

To a stirred solution of 6-methoxy-1,2,3,4-tetrahydroisoquinoline (16.3 g) in DMF (80 ml) was added K₂CO₃ (16 g) and the mixture was stirred at room temperature under nitrogen atmosphere for 10 minutes. Tert-butyl bromoacetate (14.76 ml) was added and the mixture was stirred at ambient temperature under nitrogen atmosphere for 18 hours. The mixture was poured into water (350 ml) and was extracted with 3×100 ml portions of ethyl acetate. The organic extracts were washed with water and dried over Na₂SO₄ and concentrated in vacuo to give the desired product (18.9 g) as oily gum. ¹H NMR was consistent with the proposed structure.

STEP 2

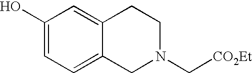

A solution of the product from STEP 1 (18.5 gm) in 48% HBr (60 ml) and glacial acetic acid (60 ml) was heated to reflux for 5 hours and was concentrated in vacuo to yield an oily residue. The residue was washed several times with diethyl ether to afford brown solid. The solid was mixed with toluene and was concentrated in vacuo to give brown solid residue. The residue was dissolved in 4N HCl in ethanol and was stirred at ambient temperature for 18 hours. The mixture was concentrated to give an oily residue which was extracted with ethyl acetate, washed with water and 5% sodium bicarbonate, dried over Na₂SO₄ and concentrated in vacuo to yield the product (7.1 g) as clear viscous oil. ¹H NMR was consistent with the proposed structure.

STEP 3

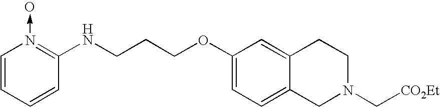

This compound was prepared in the same manner as described in EXAMPLE 2, STEP 4 replacing the product of EXAMPLE 2, STEP 3 with the product of EXAMPLE 3, STEP 2. The ¹H NMR spectrum was consistent with the proposed structure.

STEP 4

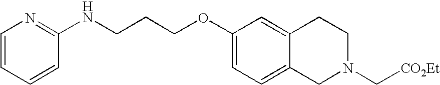

This compound was prepared following the procedure described in EXAMPLE 2, STEP 5, replacing the product of EXAMPLE 2, STEP 4 with the product of EXAMPLE 3, STEP 3. ¹H NMR spectrum was consistent with the proposed structure.

STEP 5

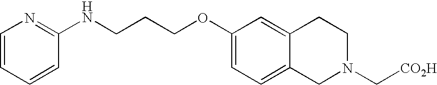

1,2,3,4-tetrahydro-6-[3-(2-pyridinylamino)propoxy]-2-isoquinolineacetic Acid, bis(trifluoroacetate)

This compound was prepared following the procedure described in EXAMPLE 2, STEP 6, replacing the product OF EXAMPLE 2, STEP 5 with the product of EXAMPLE 3, STEP 4. ¹H NMR spectrum was consistent with the proposed structure. Anal Calcd for C₁₉H₂₃N₃O₄.2.5TFA, 1H₂O: C, 43.64; H, 4.20; N, 6.36; Found: C, 43.30; H, 4.06; N, 6.24.

### EXAMPLE 4

### 1,2,3,4-tetrahydro-6-[3-(2-pyridinylamino)propoxy-2-isoquinolinepropanoic Acid

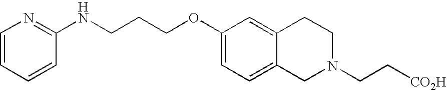

STEP 1

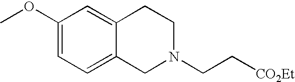

This compound was prepared following the procedure described in EXAMPLE 3, STEP 1, replacing tert-butyl bromoacetate with ethyl bromopropionate. ¹H NMR spectrum was consistent with the proposed structure.

STEP 2

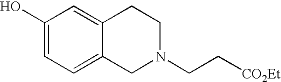

This compound was prepared following the procedure described in EXAMPLE 3, STEP 2, replacing the product of EXAMPLE 3, STEP 1 with the product of EXAMPLE 4, STEP 1. ¹H NMR spectrum was consistent with the proposed structure.

STEP 3

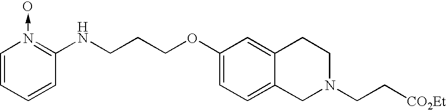

This compound was prepared following the procedure described in EXAMPLE 2, STEP 4, replacing the product of EXAMPLE 2, STEP 3 with the product of EXAMPLE 4, STEP 2. ¹H NMR spectrum was consistent with the proposed structure.

STEP 4

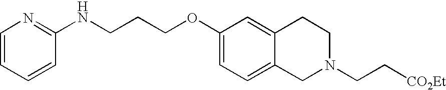

This compound was prepared following the procedure described in EXAMPLE 2, STEP 5, replacing the product of EXAMPLE 2, STEP 4 with the product of EXAMPLE 4, STEP 3. ¹H NMR spectrum was consistent with the proposed structure.

STEP 5

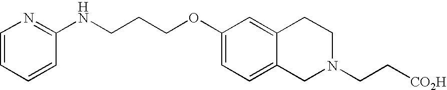

1,2,3,4-tetrahydro-6-[3-(2-pyridinylamino)propoxy-2-isoquinolinepropanoic Acid

This compound was prepared following the procedure described in EXAMPLE 2, STEP 6, replacing the product of EXAMPLE 2, STEP 5 with the product of EXAMPLE 4, STEP 4. ¹H NMR(CD₃OD): 8.18 (dd, 1H), 7.25 (m, 1H), 6.95 (m, 1H), 6.9 (d, 1H), 6.72 (m, 1H), 6.62 (m, 1H), 6.55 (m, 1H), 4.05 (t, 2H), 3.62 (s, 2H), 3.5 (m, 2H), 3.40 (s, 2H), 2.85 (m, 4H), 2.15 (m, 2H); Anal Calcd for C₂₀H₂₅N₃O₃₀.2TFA, 1.5H₂O: C, 47.22; H, 4.95; N, 6.88; Found: C, 47.05; H, 4.72; N, 6.70.

### EXAMPLE 5

### {5-[3-(pyridin-2-ylamino)propoxy]-1H-indol-1-yl}acetic Acid

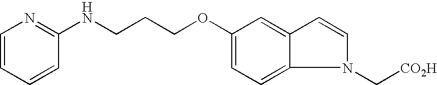

STEP 1

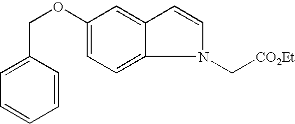

To a stirred suspension of 60% NaH (900 mg), in DMF (25 ml) was added 5-benzyloxyindole (4.46 g), in portions over 10 minutes under nitrogen atmosphere. The mixture was stirred at room temperature for 20 minutes. Ethyl bromoacetate (3.36 g) was added via syringe. The mixture was stirred at ambient temperature under nitrogen atmosphere for 1 hour. The mixture was quenched with water and was extracted with 3×50 ml portions of ethyl acetate. The organic extracts were washed with water, dried over Na₂SO₄ and concentrated in vacuo to give a clear viscous oil (5.25 g). ¹H NMR spectrum was consistent with the proposed structure.

STEP 2

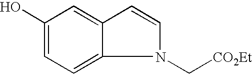

A solution of the product of the STEP 1 (5.25 g) in ethanol was shaken in Parr hydrogenation apparatus with 4% Pd on carbon under 60 psi of hydrogen pressure at ambient temperature for 16 hours. The mixture was filtered and the filtrate was concentrated to give the product (4.8 g) as a thick liquid. ¹H NMR was consistent with the proposed structure.

STEP 3

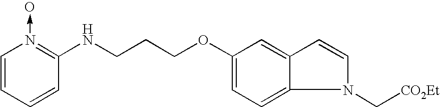

This compound was prepared following the procedure described in EXAMPLE 2, STEP 4, replacing the product of EXAMPLE 2, STEP 3 with the product of EXAMPLE 5, STEP 2. ¹H NMR spectrum was consistent with the proposed structure.

STEP 4

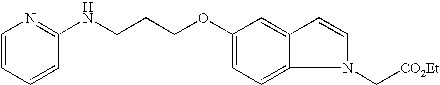

This compound was prepared following the procedure described in EXAMPLE 2, STEP 5, replacing the product of EXAMPLE 2, STEP 4 with the product of EXAMPLE 5, STEP 3. ¹H NMR spectrum was consistent with the proposed structure.

STEP 5

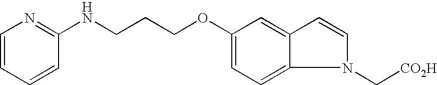

This compound was prepared following the procedure described in EXAMPLE 2, STEP 6, replacing the product of EXAMPLE 2, STEP 5 with the product of EXAMPLE 5, STEP 4. ¹H NMR spectrum was consistent with the proposed structure. Anal Calcd for C₁₈H₁₉N₃O₃.1.25TFA, 0.5H₂O: C, 51.63; H, 4.49; N, 8.81, Found: C, 51.73; H, 4.26; N, 8.94.

### EXAMPLE 6

### 2,3-dihydro-5-[3-(2-pyridinylamino)propoxy]-1H-indene-2-acetic Acid

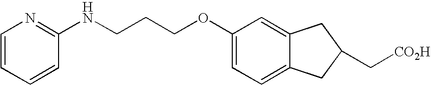

STEP 1

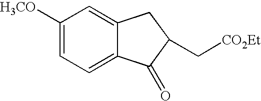

In a flame dried flask under nitrogen was placed a solution of 5-methoxy-1-indanone (3.5 g) in THF (15 mL) and chilled to zero degrees. A solution of lithiuim diisopropylamide (12.5 mL) (2.0 M in THF) was added dropwise and stirring was continued for 30 minutes. A solution of ethyl bromoacetate (5.0 g) in THF (10 mL) was rapidly added and the reaction was allowed to warm to room temperature. The reaction mixture was then partitioned between ethyl acetate and 1N HCl and the layers were separated. The aqueous portion was extracted with ethyl acetate and the combined organic extracts were washed with water, brine, dried over Na₂SO₄, concentrated and the residue purified on a silica gel column eluting with 30% ethyl acetate/hexane to produce a golden oil (1.8 g). ¹H NMR was consistent with the proposed structure.

STEP 2

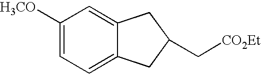

A solution of the product from STEP 1 (1.8 g) in ethanol (25 mL) containing 3 drops of phosphoric acid was shaken in a Parr hydrogenation apparatus with 20% Pd (OH)₂ on carbon under 60 psi hydrogen pressure at room temperature for 16 hours. The reaction mixture was then filtered and concentrated and the residue was purified on a silica gel column eluting with 10% ethyl acetate/hexane to afford a colorless liquid (975 mg). ¹H NMR was consistent with the proposed structure.

STEP 3

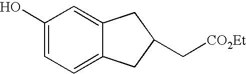

To a solution of the product from STEP 2 (970 mg) in methylene chloride (20 mL) was added boron tribromide (10 mL) (1.0 M solution in CH₂Cl₂) over 10 minutes at room temperature. After stirring for 1 hour the reaction was quenched with ethanol (5 mL) and concentrated. The residue was portioned between ethyl acetate of 10% NaHCO₃ solution. The aqueous portion was extracted with additional solvent and the combined organic extracts were washed with brine, dried over Na₂SO₄, concentrated, and the residue purified on a silica gel column eluting with 25% ethyl acetate/hexane to afford an oil (720 mg). ¹H NMR spectra was consistent for the proposed structure.

STEP 4

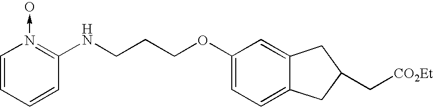

To a solution of the product from STEP 3 (704 mg) in THF (15 mL) under nitrogen was added 2-[2-hydroxy-1-propyl) amino] pyridine N-oxide (589 mg) and triphenylphosphine (918 mg). The solution was stirred at room temperature for several minutes and then a solution of diethyl azodicarboxylate (610 mg) in THF (5 mL) was added dropwise. The reaction was stirred for 18 hours and the solvent was removed in vacuo. The residue was purified on a silica gel column eluting with 96.5%.CH₂Cl₂-3.0% CH₃OH-0.5% NH₄OH to produce a golden oil (720 mg). ¹H NMR was consistent for the proposal structure.

STEP 5

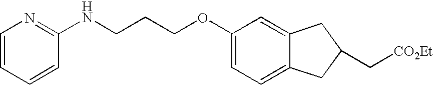

A mixture of the product from STEP 4 (700 mg), 10% Pd on carbon (400 mg), cyclohexene (3.5 mL) and isopropanol (10 mL) was refluxed for 8 hours under nitrogen. The reaction was cooled, filtered through a pad of celite and washed with excess isopropanol. The filtrate was concentrated and the residue was purified on a silica gel column eluting with 96.5% CH₂Cl₂-3.0% CH₃OH-0.5% NH₄OH to afford a viscous oil (320 mg). The ¹H NMR was consistent for the proposed structure.

STEP 6

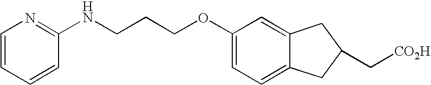

2,3-dihydro-5-[3-(2-pyridinylamino)propoxy]-1H-indene-2-acetic Acid

A solution of the product (310 mg) from STEP 5 in methanol (7.5 mL) and 1N sodium hydroxide (7 mL) was stirred at room temperature for 18 hours. The reaction was quenched with TFA (2 mL) and concentrated. The residue was purified on a reverse phase HPLC using acetonitrile/water (0.5% TFA) gradient to give a white solid (253 mg). ¹H NMR (DMSO_{d6}): 7.95 (d, 1H), 7.85 (t, 1H), 7.10 (d, 1H), 7.02 (d, 1H), 6.72 (m, 2H), 6.68 (m, 1H), 4.05 (t, 2H), 3.00 (m, 3H), 2.62 (m, 2H), 2.50 (m, 2H), 2.37 (d, 2H), 2.05 (m, 2H). Microanalysis calculated for C₁₉H₂₂N₂O₃.1.0 TFA.0.25H₂O. C, 56.69; H, 5.32; N, 6.30; Found: C, 56.91; H, 5.39; N, 6.32.

### EXAMPLE 7

### 2,3,4,5-tetrahydro-5-oxo-8-[3-(2-pyridinylamino)propoxy]-1,4-benzoxazepine-4-acetic Acid

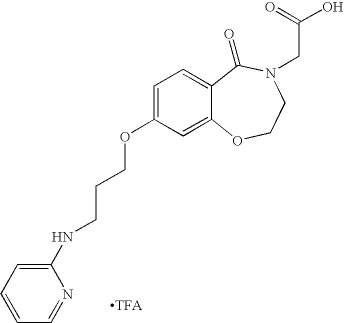

STEP 1

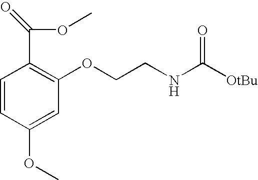

Methyl-4-methoxy salicylate (8.84 g; 48 mmole) was dissolved in THF (100 mL). To this solution was added triphenylphosphine (12.6 g; 48 mmole), and t-butyl-N-(2-hydroxyethyl) carbamate (4.68 g; 29 mmole). Diethyldiazodicarboxylate (8.4 g; 48 mmole) was added in dropwise fashion then stirred at 25° C. After 4 days, the solvent was removed and the crude residue was redissolved in ether then evaporated under reduced pressure. The crude oil was triturated with hexane twice and then resuspended in ether which caused a white precipitate to form. The precipitate was filtered away to give a crude oil (26.5 g). The crude material was purified by medium pressure chromatography (SiO₂, 50/50 ethyl acetate/hexane) to give a light yellow oil (8.5 g), which contained mostly the desired compound. ¹H NMR spectrum was consistent with the structure of the desired product.

STEP 2

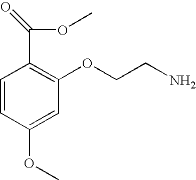

The compound produced in STEP 1 (6.06 g; 18.6 mmole) was dissolved in methylene chloride (40 mL). Trifluoroacetic acid (20 ml; 26 mmole; 29.69 g) was added and then the reaction was heated to 40-50° C. for 2-3 hours. The reaction was cooled and then the solvent was removed under reduced pressure. The resulting oil was redissolved in methylene chloride then evaporated, re-suspended in hexane and ether, evaporated, and then finally suspended in ether. The desired product precipitated out of solution as a white solid to give the desired product (5.0 g). ¹H NMR was consistent with the structure of the desired product.

STEP 3

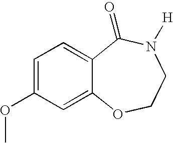

The compound produced in STEP 2 (3.1 g; 9.66 mmole) was dissolved in methanol (11.8 mL) and to this solution was added sodium bicarbonate (1.64 g). The reaction mixture was stirred at 25° C. for 1 hour then filtered. The crude residue was redissolved in THF, evaporated twice, then re-dissolved in dimethylacetamide (200 mL) and heated to 140° C. When the run was shown to be 50% complete by ¹H NMR, triethylamine (0.5 mL) was added and heating was continued for another 12 hours. Another portion of triethylamine (0.5 mL) was added. It was found that side product formation was favored after the second addition of base, thus the reaction was cooled and the solvent was removed under high vacuum. The crude brown oil was dissolved in ethyl acetate and then ether was added to the solution to precipitate out the product. The solution was filtered and the filtrates were evaporated to dryness. The resulting residue was purified through a short column (SiO₂, 100% EA) to give impure compound (800 mg) which was then washed with ether to give the desired compound (613 mg, 32% yield). ¹H NMR was consistent with the structure of the desired product.

STEP 4

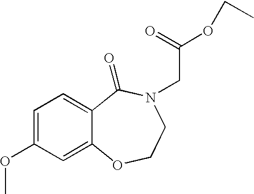

The compound produced in STEP 3 (1.5 g; 7.8 mmol) was dissolved in THF (31 mL) at 0° C. To this solution was added sodium hydride (338.3 mg; 8.5 mmol; 60% dispersion in mineral oil) and stirred for 15 minutes at 25° C. Ethyl bromoacetate (0.94 ml; 1.41 g; 8.4 mmol) was added all at once. The reaction was allowed to warm to 25° C. and stirred for 3 hours. Water was added and then this solution was extracted with methylene chloride. The organic extracts were dried (MgSO₄), evaporated and the resulting residue was purified by flash chromatography eluting with EA/hexane to give the desired product (1.36 g). ¹H NMR spectrum was consistent with the structure of the desired product.

STEP 5

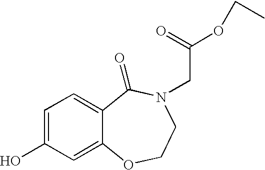

The compound produced in STEP 4 (1.36 g; 4.8 mmole) was dissolved in methylene chloride (60.1 mL) and cooled to −78° C. To this solution was added a 1M solution of boron tribromide in methylene chloride (5.6 mL) and then the solution was quickly warmed to 0° C. then stirred for 3 hours. The reaction was quenched with water and the resulting solution was extracted with CH₂Cl₂. The organic extracts were dried (MgSO₄), filtered then stripped to dryness to give the desired compound as a white crystalline solid (0.5 g). ¹H NMR spectrum was consistent with the structure of the desired product.

STEP 6

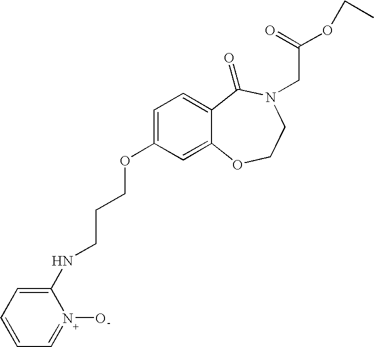

The compound produced in STEP 5 (324 mg; 1.28 mmol) was dissolved in THF (2.6 mL). N¹-(3-hydroxypropyl)-2-pyridineamine, N-oxide (215 mg; 1.28 mmole) was added to this solution which was then followed by the addition of triphenylphosphine (503.5 mg; 1.92 mmol) then (0.20 mL) diethyl diazo-dicarboxylate (222.9 mg; 1.28 mmole). A hot water bath was held under the reaction for 15 minutes, then allowed to stir for 36 hours at 25° C. The solvent was removed under reduced pressure and the crude residue was purified on a short column (SiO₂; 100% ethanol) to obtain the desired product which was obtained as a colorless oil (337 mg). ¹H NMR spectrum was consistent with the structure of the desired product.

STEP 7

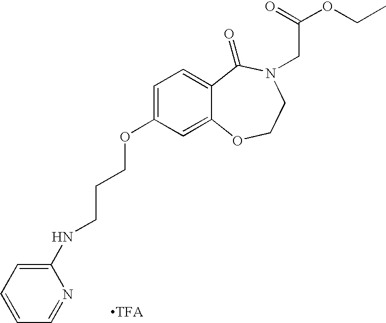

The compound produced in STEP 6 (249.9 mg; 0.58 mmol) was dissolved in isopropanol (5.8 mL) and to this solution was added 10% Pd catalyst on carbon (60.7 mg) followed by 0.58 mL of cyclohexene (476.4 mg; 5.8 mmol). The solution was heated to reflux for 2 hours. The catalyst was removed by filtration through celite. The mixture was concentrated and then the crude residue was purified by column chromatography (SiO₂, 100% ethyl acetate) to give the desired compound (140 mg). ¹H NMR spectrum was consistent with the structure of the desired product.

STEP 8

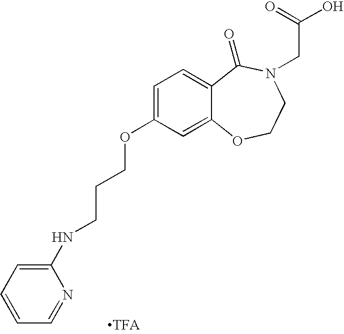

2,3,4,5-tetrahydro-5-oxo-8-[3-(2-pyridinylamino)propoxy]-1,4-benzoxazepine-4-acetic Acid, Trifluoroacetate.

The compound produced in STEP 7 was dissolved in methanol (13 mL) and to this solution was added a 1N aqueous solution of sodium hydroxide (13 mL). The reaction was stirred at 25° C. After 12 hours, trifluoroacetic acid (1.0 mL) was added to the reaction medium and then the resulting solution was evaporated to dryness. The crude sample was purified twice by reverse phase HPLC (90/10H₂O/CH₃CN/(0.5% TFA) gradient) to give the desired compound. ¹H NMR (CD₃OD) δ 7.91 (dd, 1H) 7.82 (d, 1H), 7.74 (d, 1H), 7.07 (d, 1H), 6.89 (t, 1H), 6.74 (dd, 1H), 6.58 (d, 1H), 4.49 (t, 2H), 4.36 (s, 2H), 4.19 (t, 2H), 3.70 (t, 2H), 3.60 (t, 2H), 2.21 (p, 2H); Microanalysis calculated for C₁₉H₂₁N₃O₅: C: 47.22; H: 4.25; N: 7.44. Found C: 47.23; H: 4.04; N: 7.22 1.6 TFA+0.6H₂O.

### EXAMPLE 8

### 2,3,4,5-tetrahydro-8-[3-(2-pyridinylamino)propoxy] 1,4-benzoxazepine-4-acetic Acid

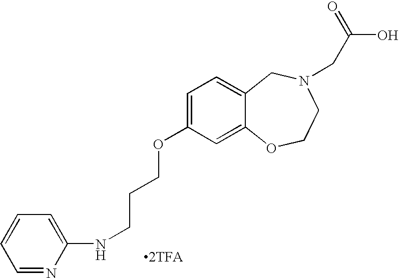

STEP 1

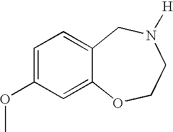

Compound 2,3,4,5-tetrahydro-8-methoxy-1,4-benzoxazepine (1.54 g; 9.4 mmol) was dissolved in THF (34 mL). To this solution was added lithium aluminum hydride (9.4 mL) (1M solution in THF). The reaction mixture was stirred at 25° C. After 12 hours, additional lithium aluminum hydride was added till the TLC (SiO₂ 25% i-propyl alcohol/ethylacetate) indicated the reaction was complete. Water (0.46 mL) then sodium fluoride (1.44 g) was added to the reaction then stirred at 25° C. for 1 hour. The solution was filtered and the filtrate was evaporated under reduced pressure to provide the desired compound as a yellow oil (1.78 g). The material was used without further purification. ¹H NMR spectrum was consistent with the structure of the desired product.

STEP 2

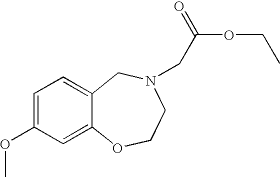

The compound produced in STEP 1 (1.25 g; 6.9 mmole) was dissolved in THF (12 mL). To this solution was added potassium carbonate (1.931 g) followed by the addition of ethyl bromoacetate (944.2 mg; 5.6 mmole) at 0° C. The solution was allowed to warm to 25° C. After 2 hours, the solution was filtered and the filtrates were evaporated to dryness. The crude residue was purified by flash chromatography (SiO₂, 100% ethyl acetate) to give the desired compound (836 mg). ¹H NMR spectrum was consistent with the structure of the desired product.

STEP 3

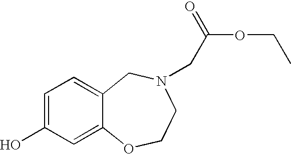

The compound produced in STEP 2 (606 mg; 2.28 mmole) was dissolved in methylene chloride (5.3 mL) and cooled to 0° C. To this solution was added a 1M solution of boron tribromide in methylene chloride (4.8 mL). After stirring for 20 minutes, the reaction was quenched with 8 ml of absolute ethanol then concentrated. The crude material was taken up in ethanol and solid sodium bicarbonate was added till all bubbling stopped. The solution was filtered and then concentrated to give a soft solid. The crude material was purified by reverse phase chromatography (95/5H₂O CH₃CN/(0.5% TFA)) gradient to give the desired compound as a colorless oil (183 mg). The free amine was formed by adding sodium bicarbonate (500 mg) which was then stirred at 25° C. for 1 hour then filtered then evaporated. The resulting material was ready to use, as is, in the next reaction step. ¹H NMR spectrum was consistent with the structure of the desired product.

STEP 4

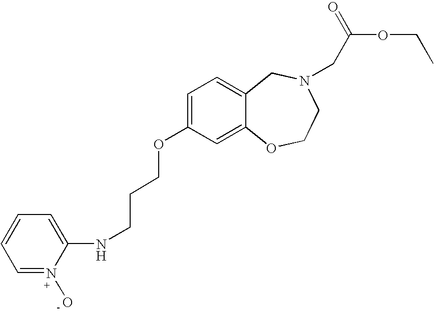

The compound produced in STEP 3 (263 mg; 1.05 mmol) and N¹-(3-hydroxy propyl)-2-pyridine amine-N-oxide (176.4 mg; 1.05 mmol) was dissolved in THF (1.9 mL). In a separate flask under an inert atmosphere triphenylphosphine and diethyldiazodicarboxylate were combined at 0° C. in THF (3.8 mL) and stirred for 10 minutes. This solution was added to the solution of the phenol and N-pyridine N-oxide then heated with a heating bath and then cooled to 25° C. After 24 hours, the solution was evaporated to dryness. The crude material was purified by reverse phase chromatography using 95/5 CH₃CN/H₂O/(0.5% TFA) by gradient elution to obtain the desired compound (217 mg). ¹H NMR spectrum was consistent with the structure of the desired product.

STEP 5

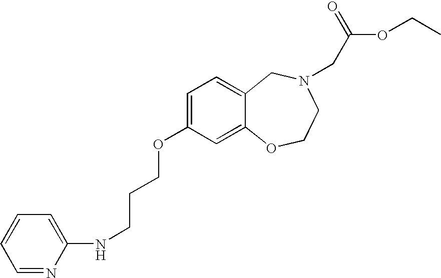

The compound produced in STEP 4 (296 mg) was dissolved in methanol (1 mL) and to this solution was added solid sodium bicarbonate (153 mg). The solution was filtered and the filtrates were stripped to dryness to give a brown oil (354.9 mg). This oil was dissolved in isopropanol (3 mL). To this solution was added 10% Pd/C (30 mg) followed by cyclohexene (0.28 mL). The solution was heated to 70-80° C. for 2-3 hours. Another 30 mg of catalyst was added then the reaction was heated for 4 more hours. The reaction mixture was filtered through celite and the filtrates were evaporated to give crude compound. The compound was purified by reverse phase chromatography to give the desired compound (354.9 mg) which was isolated as the trifluoroacetic acid salt. ¹H NMR spectrum was consistent with the structure of the desired product.

STEP 6

2,3,4,5-tetrahydro-8-[3-(2-pyridinylamino)propoxy] 1,4-benzoxazepine-4-acetic Acid

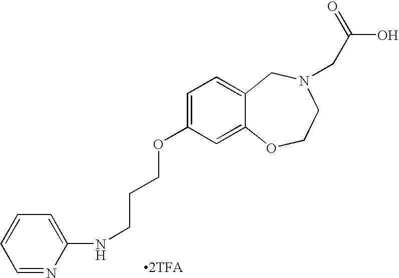

The compound produced in STEP 5 was dissolved in methanol (5 mL) and to this solution was added 1N aqueous sodium hydroxide (5 mL). The solution was stirred at 25° C. After 12 hours the solution was neutralized with trifluoroacetic acid (0.38 mL) and the resulting solution was evaporated to dryness. The crude residue was purified by reverse phase chromatography (95/5H₂O/CH₃CN (0.5% TFA) gradient elution) to give the desired compound (51 mg). ¹H NMR (CD₃OD) δ 7.89 (dd, 1H) 7.82 (d, 1H), 7.28 (d, 1H), 7.07 (d, 1H), 6.87 (t, 1H), 6.76 (dd, 1H), 6.73 (d, 1H), 4.50 (s, 2H), 4.31 (m, 2H), 4.14 (t, 2H), 4.05 (s, 2H), 3.75 (m, 2H), 3.58 (t; 2H), 2.18 (p, 2H); Microanalysis calculated for C₁₉H₂₃N₃O₄: C: 45.99; H: 4.34; N: 6.94; Found C: 46.07; H: 4.20; N: 6.84 2.1 TFA+0.5H₂O.

### EXAMPLE 9

### 6-[2-(6-Amino-2-pyridinyl)ethoxy]-1,2,3,4-tetrahydro-2-naphthaleneacetic Acid

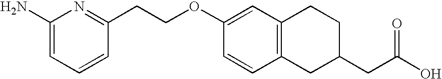

STEP 1

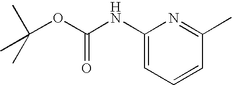

(6-methyl-2-pyridinyl)carbamic Acid, 1,1-dimethylethyl Ester

A solution of di-tert.butyl dicarbonate (0.32 g, Aldrich), 2-amino-6-picoline (15 g, Aldrich) and ether (20 mL) was allowed to stand at ambient temperature for 4 days. The volatiles were removed. The residue was purified by chromatography to give the above product as a white solid.

STEP 2

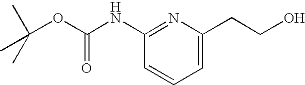

[6-(2-hydroxyethyl)-2-pyridinyl]carbamic Acid, 1,1-dimethylethyl Ester

To stirred solution of the product of Step 1 (11.9 g) in THF (100 mL) at −78° C. was added lithium diisopropylamide (85 mL, 1.5M solution in THF, Aldrich) over 5 minutes. The cooling bath was removed after 1.5 hours. The reaction mixture was cooled back to −78° C. and DMF (4.5 mL) was added. After 15 minutes, methanol (50 mL) was added followed by acetic acid (3.5 mL). Then sodium borohydride (2 g, Aldrich) was added and the reaction mixture was allowed to warm to ambient temperature. The mixture was extracted with ethyl acetate. The layers were separated. The organic phase was washed with water and concentrated in vacuo. The residue was purified by chromatography over silica gel using 20% ethyl acetate in hexane to remove starting material. Subsequent elution of the column with 60% ethyl acetate provided the above product as a white solid.

STEP 3

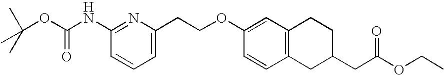

6-[2-[6-[[(1,1-dimethylethoxy)carbonyl]amino]-2-pyridinyl]ethoxy]-1,2,3,4-tetrahydro-2-naphthaleneacetic Acid, Ethyl Ester

To a stirred solution of the product of EXAMPLE 2, STEP 3 (0.259 g), the product of STEP 2 (0.3 g), triphenyl phosphine (0.33 g, Aldrich) in THF (5 mL) at −78° C. was added diisopropyl azodicarboxylate (Aldrich, 0.26 mL) over 3 minutes. The mixture was stirred at −78° C. for 3 hours and at 22° C. for 16 hours. The mixture was concentrated in vacuo and the residue was chromatographed over silica gel using 20% ethyl acetate in hexane as eluant. The fractions containing the desired product were pooled and concentrated to provide the above product as a thick gum (0.32 g).

STEP 4

6-[2-[6-[[(1,1-dimethylethoxy)carbonyl]amino]-2-pyridinyl]ethoxy]-1,2,3,4-tetrahydro-2-naphthaleneacetic Acid

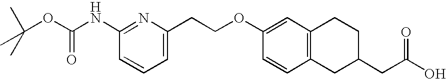

A mixture of the product of STEP 3 (0.32 g) in methanol (2 mL) and a solution of NaOH (0.7 g) in water (4.5 mL) was heated to reflux for 30 minutes. The mixture was cooled to 0° C., acidified to pH=4 and extracted with ethyl acetate. The extract was dried over MgSO₄ and concentrated in vacuo to provide the above product as a white solid.

STEP 5

6-[2-(6-Amino-2-pyridinyl)ethoxy]-1,2,3,4-tetrahydro-2-naphthaleneacetic Acid

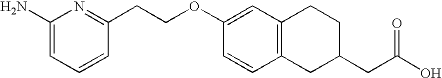

A solution of the product of STEP 4 in 4N hydrochloric acid was allowed to stir at 23° C. for 16 hours. The volatiles were removed in vacuo and the residue was washed with methanol and ether. The residue was dried in vacuo to provide the hydrochloride salt of the title product as a hygroscopic colorless solid. Microanalysis calculated for C₁₉H₂₂N₂O₃.HCl.H₂O, C 59.92; H 6.62; N 7.36; Found C 60.19; H 6.30; N 7.35.

### EXAMPLE 10

### 1,2,3,4-tetrahydro-1-oxo-6-[3-(2-tetrahydropyrimidinyl)amino]propoxy]-2-isoquinolineacetic Acid

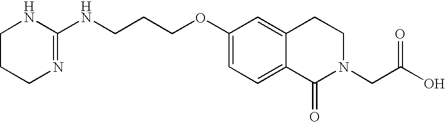

STEP 1

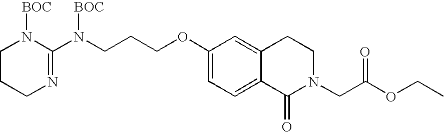

1,2,3,4-tetrahydro-1-oxo-6-[3-[2-N-Boc-tetrahydropyrimidinyl)3-N-Boc-amino]propoxy]2-isoquinolineacetate

Ethyl 1,2,3,4-tetrahydro-6-hydroxy-1-oxo-2-isoquinolineacetate (0.38 g, 1.40 mmol) was dissolved in DMF (3 mL) and triphenylphosphine (0.76 g, 7.5 mmol) was added. The solution was placed under nitrogen and cooled to 0° C. A solution of (2-N-Boc-tetrahydropyrimidinyl)-3-N-Boc-amino-propanol (1.0 g, 2.80 mmol) and DEAD (0.498 g, 2.9 mmol) in DMF (3 mL) was added slowly. The reaction mixture was stirred for 18 hours, while under nitrogen at room temperature. The solution was concentrated and carried to the next step without further purification.

STEP 2

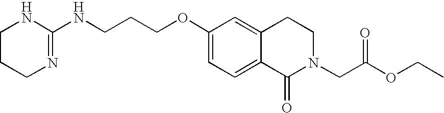

1,2,3,4-tetrahydro-1-oxo-6-[3-[(2-tetrahydropyrimidinyl)-amino]propoxy]2-isoquinolineacetate

1,2,3,4-tetrahydro-1-oxo-6-[3-[2-N-Boc-tetrahydropyrimidinyl)-3-N-Boc-amino]propyloxy]-2-isoquinolineacetate (1.5 g, 2.55 mmol) was dissolved in ethanol/HCl and stirred at 0° C. initially under nitrogen then allowed to continue for 18 hours. The reaction mixture was concentrated and purified by HPLC. ¹H NMR was consistent with the desired product. Yield: 0.8 g (80%).

STEP 3

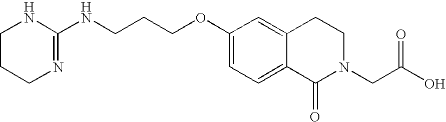

1,2,3,4-tetrahydro-1-oxo-6-[3-(2-tetrahydropyrimidinyl)amino]propoxy]-2-isoquinolineacetic Acid

1,2,3,4-tetrahydro-1-oxo-6-[3-[(2-tetrahydropyrimidinyl)amino]propoxy]-2-isoquinolineacetate (0.8 g, 2.06 mmol) was dissolved in a minimum amount of ethanol and water. Sodium hydroxide (2.5N) was added until basic and the solution was stirred at room temperature for 3 hours. The solution was concentrated under reduced pressure without heat to remove ethanol. The solution was cooled to 0° C. and TFA was added until acidic. The solution was concentrated. MS and ¹HNMR were consistent with the desired product. Yield: 0.540 g (55%).

### EXAMPLE 11

### 3,4-dihydro-7-[3-(2-pyridinylamino)propoxy]-2-H-1-benzopyran-3-acetic Acid

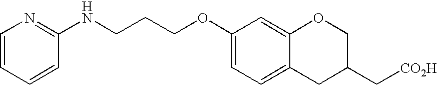

STEP 1

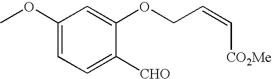

To a stirred solution of 4-methoxysalicylaldehyde (1.52 g) in DMF (12 ml) was added K₂CO₃ (1.52 g) and the mixture was stirred at room temperature under nitrogen atmosphere for 10 minutes. Methyl 4-bromocrotonate (1.3 ml) was added and the mixture was stirred at ambient temperature for one hour. The mixture was poured into water (50 ml) and was extracted with ethyl acetate (3×25 ml). The organic extracts were washed with water and dried over Na₂SO₄ and concentrated in vacuo to give the crude residue which upon crystallization from ethyl acetate afforded the desired product as a white solid (0.71 g). ¹HNMR was consistent with the proposed structure.

STEP 2

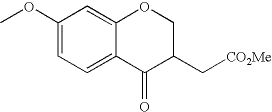

A solution of the product from STEP 1 (0.620 g) in DMF (5 ml) and 3-benzyl-5-(2-hydroxyethyl)-4-methyl-1,3-thiazolium chloride (90 mg) was heated to 130° C. under nitrogen atmosphere for 24 hours. The mixture was cooled to room temperature, was poured into water (30 ml) and was extracted with ethyl acetate (3×20 ml). The organic extracts were washed with water, followed by 1N HCl and water, and followed by a saturated solution of sodium bicarbonate. The organic extracts were dried (Na₂SO₄) and concentrated in vacuo to yield an oily residue which upon crystallization from ether afforded the desired product as a white solid (0.42 g). ¹H NMR was consistent with the proposed structure.

STEP 3

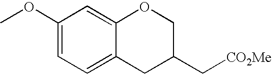

A solution of the product from STEP 2 (0.45 g) in ethanol (10 ml) containing 2 drops of phosphoric acid was shaken in a Parr hydrogenation apparatus with 20% Pd(OH)₂ on carbon under 60 psi hydrogen pressure at ambient temperature for 16 hours. The reaction mixture was filtered and concentrated and the residue was purified on silica gel column eluting with 10% ethyl acetate/hexane to afford a colorless liquid (220 mg). ¹H NMR was consistent with the proposed structure.

STEP 4

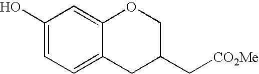

The above compound was prepared following the procedure described in EXAMPLE 2, STEP 3, and replacing the product of EXAMPLE 2, STEP 2 with the product of EXAMPLE 11, STEP 3. ¹H NMR was consistent with the proposed structure

STEP 5

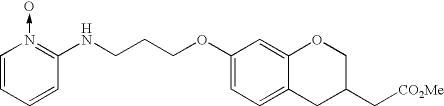

The above compound was prepared following the procedure described in EXAMPLE 2, STEP 4 and replacing the product of EXAMPLE 2, STEP 3 with the product of EXAMPLE 11, STEP 4. ¹H NMR was consistent with the proposed structure.

STEP 6

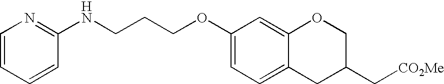

The above compound was prepared following the procedure described in EXAMPLE 2, STEP 5, and replacing the product of EXAMPLE 2, STEP 4 with the product of EXAMPLE 11, STEP 5. ¹H NMR was consistent with the proposed structure.

STEP 7

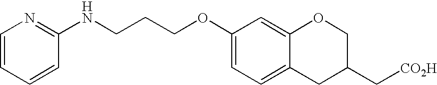

3,4-dihydro-7-[3-(2-pyridinylamino)propoxy]-2-H-1-benzopyran-3-acetic Acid

This compound was prepared following the procedure described in EXAMPLE 2, STEP 6, and replacing the product of EXAMPLE 2, STEP 5 with the product of EXAMPLE 11, STEP 6. ¹H NMR(CD₃OD): 7.85 (t, 1H), 7.80 (d, 1H), 7.06 (d, 1H), 6.9 (m, 2H), 6.45 (dd, 1H), 6.34 (d, 1H), 4.20 (dd, 1H), 4.05 (t, 2H), 3.80 (m, 1H), 3.57 (t, 2H), 2.87 (m, 1H), 2.25-2.50 (m, 4H), 2.15 (m, 2H). Anal Calcd for C₁₉H₂₂N₂O₄.1.1TFA, 0.5H₂O: C, 53.40; H, 5.09; N, 5.87; Found: C, 53.30; H, 4.89; N, 5.65.

### EXAMPLE 12

### (6-{[3-(pyridin-2-ylamino)propyl]thio}-1,2,3,4-tetrahydronaphthalen-2-yl)acetic Acid

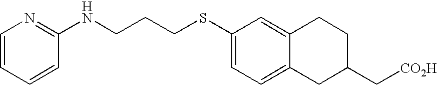

STEP 1

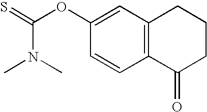

A mixture of 6-Hydroxy-1-tetralone (4.0 g), K₂CO₃ (3.5 g) and DMF (40 ml) was heated to 80° C. under nitrogen atmosphere for 30 min. N,N-dimethyl-aminothiocarbamoyl chloride (3.75 g) was added and the mixture was stirred at ambient temperature for 90 min. The mixture was added to ice water (100 ml) and was extracted with (3×30 ml) portions of ethyl acetate. The organic layer was washed with 10% NaOH solution, water, and dried over Na₂SO₄. The solid was filtered and the filtrate was concentrated. The residue was crystallized from ether to give 3.5 g of desired product as white crystalline solid. NMR spectrum of this material was consistent for the proposed structure.

STEP 2

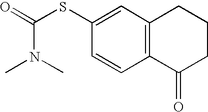

The product of STEP 1 (3.5 g) was heated to 230° C. under nitrogen atmosphere for 1 hour and was cooled to ambient temperature. The residue was crystallized from ether to yield 2.8 gm of desired product as white solid. NMR spectrum of this material was consistent for the proposed structure.

STEP 3

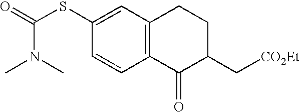

The product of STEP 2 (3.73 g, 15 mmol) was added to THF (50 ml) containing 1.5M LDA solutions in THF (12 ml) at −50° C. under nitrogen atmosphere. The reaction mixture was stirred at −50° C. under nitrogen atmosphere for 15 min. and was quenched with ethyl bromoacetate (2.0 ml). The mixture was warmed to room temperature and was stirred at room temperature for 1.5 hours. The mixture was treated with a saturated solution of ammonium chloride and was extracted with ethyl acetate. The organic extract was washed with water and dried over Na₂SO₄. The solid was filtered and the filtrate was concentrated. Reverse Phase HPLC using acetonitrile-water gradient 10-90% in 30 min to yield 1.45 g of the desired compound as oil. NMR spectrum of this material was consistent for the proposed structure.

STEP 4

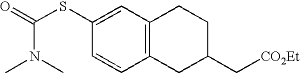

The product of STEP 3 (1.4 g) was dissolved in Ethanol (30 ml) and was treated with NaBH₄ (82 mg) and the mixture was stirred at an ambient temperature for 3 hours. The mixture was concentrated and the residue was dissolved in TFA (4.0 ml) and was treated with Et₃SiH (2.0 ml). The reaction mixture was stirred at an ambient temperature for 4 hours. The mixture was concentrated. The residue was dissolved in ethyl acetate and was washed with a saturated solution NaHCO₃. The organic layer was dried over Na₂SO₄, filtered, and concentrated. The residue was purified by silica gel chromatography using 40% EA/Hexanes as mobile phase to yield 0.58 g of the desired compound as oil. NMR spectrum of this material was consistent for the proposed structure.

STEP 5

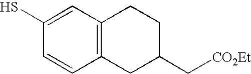

The product of STEP 4 (560 mg) was dissolved in methanol (4.0 ml) and was treated with NaOH (500 mg). The mixture was heated to reflux under nitrogen atmosphere for 2 hours. The mixture was concentrated. The residue was acidified with concentrated HCl. The acidic mixture was extracted with ethyl acetate and was washed with water. The organic layer was dried over Na₂SO₄, filtered and concentrated. The residue was dissolved in 8N ethanol HCl. The solution was stirred at room temperature for 18 hours. And concentrated. The residue was dissolved in ethyl acetate and was washed with water, dried (Na₂SO₄) and concentrated. The residue was purified by silica gel chromatography using 40% ethylacetate/hexanes as mobile phase to yield 0.189 g of the desired compound as oil. NMR spectrum of this material was consistent for the proposed structure.

STEP 6

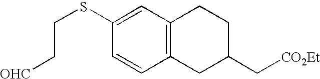

The product of STEP 5 (189 mg) was dissolved in acetonitrile (5.0 ml) and Et₃N (0.5 ml) and the mixture was treated with acrolein (150 mg). The reaction mixture was stirred at room temperature for 2 hours under nitrogen atmosphere. The mixture was concentrated and the residue was dissolved in ethyl acetate and was washed with water, and dried over Na₂SO₄. The solid was filtered and the filtrate was concentrated to afford 0.21 g of the desired product as oil. NMR spectrum of this material was consistent for the proposed structure.

STEP 7

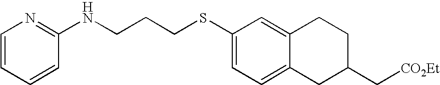

The product of STEP 6 (210 mg, 0.78 mmol) was dissolved in CH₂Cl₂ (10 ml) and was cooled to 0° C. and 2-aminopyridine (80 mg, 0.858 mmol) was added followed by the addition of sodium triacetoxyborohydride (248 mg, 1.17 mmol) under nitrogen atmosphere. The ice bath was removed and the mixture was stirred at an ambient temperature for 18 hours. The mixture was diluted with a saturated sodium bicarbonate solution (10 ml). Organic extract was washed with water, dried over Na₂SO₄, and concentrated to afford 0.2 g of the desired product as oil. NMR spectrum of this material was consistent for the proposed structure.

STEP 8

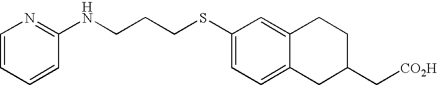

The product of STEP 7 (200 mg) was dissolved methanol (2.0 ml) and THF (2.0 ml) and was treated with 1N NaOH solution (2.0 ml). The mixture was stirred at room temperature for 1 hour, and was concentrated. The residue was neutralized with 1N HCl (2.0 ml) and concentrated. The residue was purified by Reverse Phase HPLC using acetonitrile Water gradient 5-50% in 30 min to yield 96 mg of the desired compound as an oily gum as TFA salt. ¹H NMR (CD₃OD) 7.90 (m, 1H), 7.80 (d, 1H), 7.14 (m, 2H), 6.9 (dd, 2H), 6.88 (t, 1H), 3.5 (m, 2H), 3.06 (t, 2H), 2.85 (m, 1H), 2.78 (m, 2H), 2.42 (m, 1H), 2.35 (d, 2H), 2.15 (m, 1H), 2.01 (m, 3H). 1.44 (m, 1H); Anal. Calcd. For C₂₀H₂₄N₂SO₃ plus 1.25 CF₃CO₂H, plus 1H₂O: C, 52.27; H, 5.31; N, 5.42. Found: C, 452.62; H, 5.29; N, 5.48; Mass Spectrum: (MH⁺)=357.2

### EXAMPLE 13

### 6-{(Pyridin-2-yl)-3-amino-1-propyloxy}2-quinolinylacetic Acid

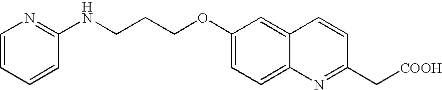

STEP 1

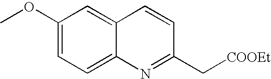

Ethyl 6-methoxy-2-quinolylacetate.

Ethyl acetoacetate (10.0 g) was added to 6-methoxyquinoline-1-oxide (10.0 g, 57.14 mmol) in acetic anhydride (15 mL) and the reaction mixture was heated at 40° C. for 6 h. The reaction mixture was poured in to an ice-cold solution of 10% HCl (100 mL). After 1 h, the reaction mixture was extracted with dichloromethane (2×100 mL). The combined organic extracts were dried and concentrated to afford 4.5 g (32%) of the desired product as oil. ¹H NMR (CD₃OD) δ 8.20 (d, 1H, J=8.5 Hz), 7.86 (d, 1H, J=9.4 Hz), 7.44 (d, 1H, J=8.6 Hz), 7.37 (m, 1H), 7.24 (d, 1H, J=8.9 Hz), 4.16 (q, 2H, J=7.1 Hz), 3.97 (s, 2H), 3.91 (s, 3H), 1.23 (t, 3H, J=7.1 Hz). Anal. Calcd for C₁₄H₁₅NO₃: 246.1130 (M+H). Found, Mol. Wt, 246.1144 (M+H, HRMS).

STEP 2

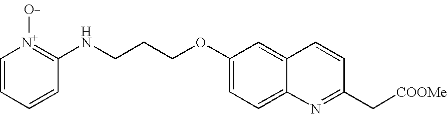

Methyl 6-{(pyridin-2-yl)-3-amino-1-propyloxy}2-quinolinylacetate.

Boron tribromide (3.08 g) was added to a solution of ethyl 6-methoxy-2-quinolylacetate (1.50 g, 6.12 mmol) in dichloromethane (25 mL) at 0° C. and was stirred 18 h, then quenched with methanol. The reaction mixture was concentrated to afford 0.81 g of the desired product as an oil. A solution of DEAD (1.30 g) and N-(2-pyridyl-N-oxide)-3-aminopropanol (1.26 g) in DMF (10 mL) was added to a solution of methyl 6-hydroxyquinolinyl-2-acetate (0.81 g, 3.73 mmol) and triphenylphosphine (2.15 g) in DMF (15 mL) over a period of 5 min and the reaction mixture was stirred for 24 h. DMF was removed in vacuo and the residue was purified by hplc (reverse phase C18, 10%-100% gradient of acetonitrile in water containing 0.05% TFA) to afford 0.60 g (44%) of the desired product as an oil. ¹H NMR (CD₃OD) δ 8.87 (m, 1H), 8.15 (m, 1H), 8.08 (m, 1H), 7.8-7.94 (m, 2H), 7.76 (m, 1H), 7.67 (m, 1H), 7.17 (d, 1H, J=9 Hz), 6.78 (m, 1H), 4.32 (t, 2H, J=5.9 Hz), 3.76 (s, 3H), 3.69 (t, 2H, J=6.6 Hz), 2.26 (t, 2H, J=5.9 Hz). Anal. Calcd for C₂₀H₂₁N₃O₄: Mol. Wt, 367.1532. Found: Mol. Wt, 368.1634 (M+H, HRMS).

STEP 3

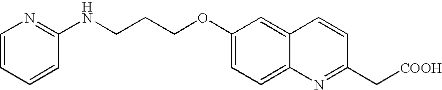

6-{(pyridin-2-yl)-3-amino-1-propyloxy}2-quinolinylacetic Acid

A mixture of Methyl 6-{(1-oxopyridin-2-yl)amino-1-propyloxy}2-quinolinyl-acetate (0.60 g), palladium/C (0.20 g), cyclohexene (2 mL) in ethanol (30 mL) was heated at reflux over 24 h. The reaction mixture was filtered, and the residue was washed with additional amount of ethanol (100 mL). The combined filtrates were concentrated. The residue was added ethanol (5 mL) and sodium hydroxide (5 mL, 2.5 N) and stirred for 8 h. The reaction mixture was concentrated and the residue was dissolved in water (5 mL) and the pH was adjusted to 2 by the addition of TFA. This was purified by hplc (reverse phase C18, 10%-100% gradient of acetonitrile in water containing 0.05% TFA) and 0.50 g (68%) of the desired product was obtained as its TFA salt. ¹H NMR (CD₃OD) δ 8.87 (m, 1H), 8.08 (m, 1H), 7.8-7.95 (m, 2H), 7.7-7.8 (m, 1H), 7.76 (m, 1H), 7.67 (m, 1H), 7.17 (d, 1H, J=9 Hz), 6.86 (t, 1H, J=6.6 Hz), 4.35 (m, 2H), 3.63 (m, 2H), 2.28 (m, 2H). Anal. Calcd for C₁₉H₁₉N₃O₃: Mol. Wt, 337.1426. Found: Mol. Wt, 294.1632 (M+H-CO2, HRMS).

### EXAMPLE 14

### 6-{(Pyridin-2-yl)-3-amino-1-propyloxy}1,2,3,4-tetrahydro-isoquinoline-1-oxo-2-acetic Acid

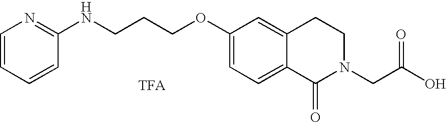

STEP 1

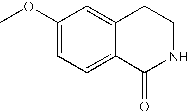

3,4-Dihydro-6-methoxy-1(2H)-isoquinolinone.

5-Methoxyindan-1-one (1.0 g, 6.17 mmol) was placed in round bottom flask. Methanesulfonic acid (10 mL) was added and placed under nitrogen and cooled to 0° C. Sodium azide (0.42 g, 6.8 mmol) was added slowly over 0.5 hour, while the solution was cooled and stirred. The solution was allowed to stir for 2 hours. The solution was quenched with water and extracted with dichloromethane (3×50 mL). The organic layer was washed with sodium bicarbonate solution and brine. The solution was dried over magnesium sulfate, filtered, and concentrated. The resulting residue was triturated with ether and slight amount of ethyl acetate to afford 0.300 g (27%) of the desired product. ¹H NMR (DMSO-d₆) δ 7.76 (m, 1H), 6.87 (m, 2H), 3.79 (s, 3H), 3.33 (m, 2H), 2.85 (m, 2H). Anal. Calcd for C₁₀H₁₁NO₂: 178.0868 (M+H). Found: Mol. Wt, 178.0859 (M+H, HRMS).

STEP 2

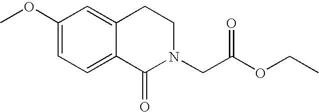

Ethyl 1,2,3,4-tetrahydro-6-methoxy-1-oxo-2-isoquinolineacetate

Sodium hydride (39.6 mg, 1.65 mmol) was washed with hexane three times for removal of oil. A solution of 3,4,dihydro-6-methoxy-1 (2H)-isoquinolinone (267 mg, 1.5 mmol) in THF (10 mL) was added to sodium hydride (0.0396 g, 1.65 mmol). The solution was refluxed for 1 hour, then cooled to room temperature. The reaction mixture was then treated with ethyl bromoacetate (0.276 g, 1.65 mmol) and stirred for 18 hours. Water was added to quench the reaction and extracted with ethyl acetate (3×50 mL). The combined extracts were dried over magnesium sulfate, and concentrated. The material was recrystallized from hexane to afford 0.094 g (24%) of the desired product. ¹H NMR (DMSO-d₆) δ 7.76 (m, 1H), 6.86 (m, 2H), 4.23 (s, 2H), 4.08 (m, 2H), 3.79 (s, 3H), 3.58 (m, 2H), 2.97 (m, 2H), 1.18 (m, 3H). Anal. Calcd for C₁₄H₁₇NO₄: Mol. Wt, 264.1236 (M+H). Found: Mol. Wt, 264.1242 (M+H, HRMS).

STEP 3

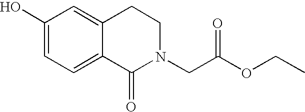

Ethyl 1,2,3,4-tetrahydro-6-hydroxy-1-oxo-2-isoquinolineacetate

Ethyl 1,2,3,4-tetrahydro-6-methoxy-1-oxo-2-isoquinolineacetate (0.094 g, 0.357 mmol) was dissolved in dichloromethane (2 mL) and cooled under nitrogen to 0° C. Boron tribromide (0.090 g, 0.357 mmol) was added and the reaction mixture was stirred for 18 hours and quenched with ethanol. The solution was concentrated to afford 0.07 g (78%) of the desired product. ¹H NMR (DMSO-d₆) δ 7.68 (m, 1H), 6.61-6.70 (m, 2H), 4.21 (s, 2H), 4.10 (m, 2H), 3.52 (m, 2H), 3.42 (m, 2H), 1.18 (m, 3H). Anal. Calcd for C₁₃H₁₅NO₄: Mol. Wt, 250.1079 (M+H). Found: Mol. Wt, 250.1074 (M+H, HRMS).

STEP 4

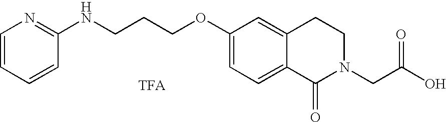

6-{(Pyridin-2-yl)-3-amino-1-propyloxy}1,2,3,4-tetrahydroisoquinoline-1-oxo-2-acetic Acid.

Ethyl 1,2,3,4-tetrahydro-6-hydroxy-1-oxo-2-isoquinolineacetate (1.2 g, 4.8 mmol) was dissolved in DMF (9 mL) and place under nitrogen. Triphenylphosphine (2.61 g, 9.9 mmol) was added and the solution was cooled to 0° C. The solution of N-oxide (1.62 g, 9.6 mmol) was dissolved in DMF (9 mL) with slight heating, then cooled. DEAD (1.71 g, 9.8 mmol) was added to the N-oxide solution. The N-oxide solution was slowly added to first solution while at 0° C. The reaction mixture was allowed to stir at room temperature for 3 hours then concentrated and purified by HPLC. The product obtained (0.8 g, 2.0 mmol) was dissolved in ethanol and Pd/C (10%, 0.45 g) was added and placed under nitrogen. Cyclohexene (1.0 g, 12.2 mmol) was added and the reaction mixture was refluxed under nitrogen for 20 hours. The catalyst was filtered off and the filtrate was concentrated. The resulting residue was dissolved in water with minimum amount of ethanol. Sodium Hydroxide 2.5N was added until basic and allowed to stir for 2 hours. Concentrated under reduced pressure to remove ethanol, and then cooled to 0° C. TFA was added until acidic, then the solution was concentrated. The crude product was purified by HPLC. Yield: 0.206 g. ¹H NMR (DMSO-d₆) δ 7.81-7.94 (m, 3H), 6.81-7.02 (m, 4H), 4.21 (m, 4H), 3.40-3.62 (m, 4H), 2.90 (m, 2H), 2.10 (m, 2H). Anal. Calcd for C₁₉H₂₁N₃O₄: Mol. Wt, 356.1610 (M+H). Found: Mol. Wt, 356.1609 (M+H, HRMS).

### EXAMPLE 15

### 1,2,3,4-tetrahydro-6-[2-(5,6,7,8-tetrahydro-1,8-naphthyridyl)-aminoethyloxy]-2-naphthaleneacetic Acid

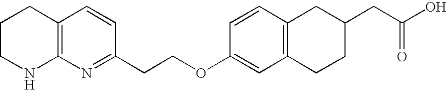

STEP 1

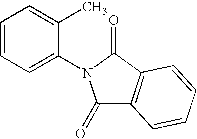

2-(3-methyl-2-pyridinyl)1H-isoindole-1,3 (2H)-dione

To a neat 2-amino-3-picoline (91 g, 0.84 mol) was added phthalic anhydride (125 g, 0.84 mol), the resulting solid mixture was heated at 120° C. and water was distilled off from the reaction mixture. The reaction mixture was cooled to room temperature and solid was dissolved in dichloromethane (1 L). The organic solution was washed with water (2×500 mL), brine (1×500 mL), dried over MgSO₄. The colored solution was treated with activated carbon, filtered and filtrate was concentrated under reduced pressure. Ether (300 mL) was added to the concentrated residue and stirred at room temperature overnight. The solid was filtered and washed with ether, and dried to give 176 g (88%) white solid. ¹H NMR (DMSO) δ 8.44-8.46 (m, 1H), 7.88-8.01 (m, 5H), 7.46-7.49 (m, 1H), 2.17 (m, 3H). Mol. Wt: 239.19 (M+H).

STEP 2

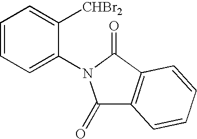

2-[3-(dibromomethyl)-2-pyridinyl]-1H-isoindole-1,3(2H)-dione

To a suspension solution of 2-(3-methyl-2-pyridinyl)1H-isoindole-1,3 (2H)-dione (14.6 g, 61 mmol) and NBS (25 g, 140 mmol) in CCl₄ (160 mL) was added AlBN (0.1 g), the reaction mixture was refluxed and a irradiated with a sun lamp. AlBN (0.1 g) were added every 30 minutes until the starting material was all consumed. The mixture was cooled to room temperature and the solid was filtered. The solid was taken up to dichloromethane (400 mL) and washed with 5% Na₂S₂O₃ (3×150 mL), water (1×150 mL) and dried over Na₂SO₄. The solvent was removed under reduced pressure. The concentrated solid was suspended in ether. Solid was filtered and dried to give 20.5 g (84.5%) yellow solid. ¹H NMR (DMSO) δ 8.63-8.64 (m, 1H), 8.51-8.55 (m, 1H), 7.92-8.01 (m, 4H), 7.74-7.78 (m, 1H) 7.6 (s, 1H). Mol. Wt: 396.92 (M+H).

STEP 3

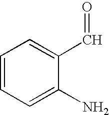

2-amino-3-pyridinecarboxaldehyde. {A. E. Moorman, S. Yu. Synthetic communications, 17,1695-1699 (1987)}

To a solution of 2-[3-(dibromomethyl)-2-pyridinyl]-1H-isoindole-1,3(2H)-dione (20 g, 50 mmol) in ethanol (250 mL) was added conc. NH₄OH (25 mL) at 4° C. The reaction mixture was stirred 10 minutes at 4° C. then stirred at room temperature for one hour. Reaction mixture was concentrated under reduced pressure. To the concentrated residue was added con. HCl (150 mL) and mixture was refluxed for 3 hours. The reaction mixture was cooled to room temperature and concentrated. To the concentrated residue was added water (25 mL) then added saturated K₂CO₃ to neutralize the solution. The solution was extracted with dichloromethane (3×150 mL). The combined organic solution was washed with water (3×150 mL), brine (1×200 mL), dried over Na₂SO₄. The solid was filtered and the filtrate was concentrated. The concentrated residue was suspended in ether, filtered and washed with ether to give 4.3 g (70%) yellow solid. ¹H NMR (DMSO) δ 9.82 (s, 1H), 8.20-8.22 (m, 1H), 7.95-7.98 (m, 1H), 7.51 (bs, 2H), 6.69-6.73 (m, 1H).

STEP 4

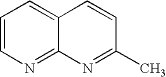

2-methyl-1,8-naphthyridine. (E. M. Hawes, D. G. Wibberly. J. Chem. Soc(C). 1966, 315)

To a solution of 2-amino-3-pyridinecarboxaldehyde (2 g, 16 mmol) in ethanol (3 mL) was added acetone (1.9 g, 32 mmol) and piperidine (0.34 g, 4 mmol) and the reaction mixture was refluxed 24 hours. Reaction mixture was cooled to room temperature then concentrated in vacuum. Ether was added to concentrated residue. The solid was filtered and dried to give 1.62 g (69%) yellow solid. ¹H NMR (CD₃OD) δ 8.39-8.99 (m, 1H) 8.36-8.39 (m, 1H), 8.30 (d, 2H, J=8.33 Hz), 7.52-7.58 (m, 2H), 2.76 (s, 3H).

STEP 5

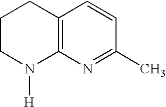

To a solution of 2-methyl-1,8-naphthyridine (2 g, 13.9 mmol) in ethanol (35 mL) was added 10% Pd/C, and the reaction mixture was stirred under H₂ (10 psi) for 24 hours. Palladium was filtered out through celite and washed with excess ethanol. The filtrate was concentrated under vacuum to give 1.7 g (83%) pink solid. ¹H NMR (CD₃OD) δ 7.07 (d, 1H, J=7.38 Hz) 6.32 (d, 1H, J=7.25 Hz), 3.33-3.36 (m, 2H), 2.65-2.76 (m, 2H), 2.22 (s, 3H), 1.82-1.87 (m, 2H). Mol. Wt: 149.15 (M+H).

STEP 6

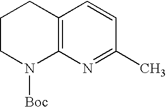

2-methyl-8-(tert-butoxycarbonyl)-5,6,7,8-tetrahydro-1,8-naphthyridine

To a solution of 2-methyl-5,6,7,8-tetrahydro-1,8-naphthyridine (1 g, 6.7 mmol) in dichloromethane (10 mL) was added di-tert-butyl dicarbonate (3 g, 13 mmol), triethylamine (0.68 g, 6.7 mmol) and 4-DMAP (50 mg), the reaction mixture was refluxed overnight. The reaction mixture was concentrated under vacuum. The concentrated residue was purified on silica gel (1% methanol in dichloromethane) to give 1.1 g (69%) orange solid. ¹H NMR (CD₃OD) δ 7.44 (d, 1H, J=7.76 Hz) 6.95 (d, 1H, J=7.76 Hz), 3.72-3.76 (m, 2H), 3.29-3.31 (m, 2H), 2.73-2.78 (m, 2H), 2.43 (s, 3H), 1.88-1.95 (m, 2H), 1.50 (s, 9H).

STEP 7

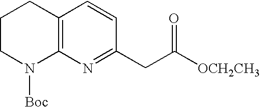

Ethyl [8-(tert-butoxycarbonyl)-5,6,7,8-tetrahydro-1,8-naphthyridin-2-yl]-acetate

To a solution of 2-methyl-8-(tert-butoxycarbonyl)-5,6,7,8-tetrahydro-1,8-naphthyridine (1.4 g, 5.6 mmol) and diethyl carbonate (2.5 g, 20 mmol) in THF (10 mL) was added LDA (8 mL of 2M solution in hexane) at −78° C. and stirred at −78° C. for 40 minutes. The reaction was quenched with saturated NH₄Cl and extracted with ethyl acetate (2×100 mL). Combined organic solution was concentrated and purified on silica gel column to give 1.5 g (83%) yellow oil. ¹H NMR (CD₃OD) δ 7.39 (d, 1H, J=7.47 Hz), 7.0 (d, 1H, J=7.61 Hz), 4.10-4.20 (m, 2H), 3.74-3.80 (m, 4H), 2.75 (t, 2H, J=6.59 Hz), 1.88-1.97 (m, 2H), 1.51 (s, 9H), 1.25 (t, 3H, J=7.10 Hz).

STEP 8

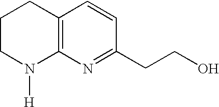

2-(5,6,7,8-tetrahydro-1,8-naphthyridin-2-yl)-1-ethanol (WO 0033838).

To a solution of ethyl [8-(tert-butoxycarbonyl)-5,6,7,8-tetrahydro-1,8-naphthyridin-2-yl]acetate (3.8 g, 11.8 mmol) in THF (20 mL) was added LiBH₄ (7.6 mL of 2M solution in hexane, 15.2 mmol), the reaction was refluxed overnight. The reaction mixture was cooled in ice bath and quenched with water. The mixture was extracted with ethyl acetate (3×50 mL). The combined organic solution was dried over MgSO4, concentrated and dried under vacuum to give 2.9 g oil. The oil was taken up in dichloromethane (10 mL) and 4N HCl in dioxane (10 mL) was added. The solution was stirred 4 hours at room temperature then concentrated under vacuum. To the concentrated residue was added 1:1/1N NaOH:brine (50 mL) and extracted with dichloromethane (3×80 mL). The combine organic solution was concentrated and purified on silica gel to give 1 g (47%) oil. ¹H NMR (CD₃OD) δ 7.10 (d, 1H, J=7.38 Hz), 6.36 (d, 1H, J=7.38 Hz), 3.77 (t, 2H, J=6.84 Hz), 3.45 (t, 2H, J=5.57 Hz), 2.66-2.71 (m, 4H), 1.82-1.88 (m, 2H).

STEP 9

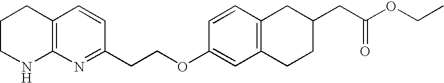

Ethyl 1,2,3,4-tetrahydro-6-[2-(5,6,7,8-tetrahydro-1,8-naphthyridyl)aminoethyloxy]-2-naphthaleneacetate

A solution of 2-(5,6,7,8-tetrahydro-1,8-naphthyridyl)-aminoethanol (0.285 g, 1.85 mmol) and DEAD (0.644 g, 3.70 mmol) in DMF (5 mL) was added to a solution of ethyl 6-hydroxy-5,6,7,8-tetrahydronaphthalene-2-acetate (0.40 g, 1.85 mmol) and triphenylphosphine (0.969 g, 3.70 mmol) in dimethylformamide (10 mL) and was stirred for 18 h at rt. The solvent was removed and the residue was purified by hplc to afford 0.10 g (15%) of the desired product. ¹H NMR δ 7.57 (d, 1H, J=7.4 Hz), 6.89 (d, 1H, J=8.3 Hz), 6.69 (d, 1H, J=7.4 Hz), 6.59-6.62 (m, 2H), 4.20 (t, 2H, J=5.9 Hz), 4.12 (m, 2H), 3.47 (t, 2H, J=5.8 Hz), 3.1 (t, 2H, J=5.9 Hz), 2.73-2.81 (m, 5H), 2.32-2.38 (m, 3H), 2.13 (m, 1H), 1.93 (m, 3H), 1.40 (m, 1H), 1.24 (t, 3H, J=7.3 Hz). Anal. Calcd for C₂₄H₃₀N₂O₃: Mol. Wt, 394.2256 Found: Mol. Wt, 395.2339 (M+H, HRMS).

STEP 10

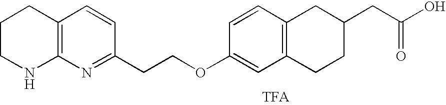

1,2,3,4-tetrahydro-6-[2-(5,6,7,8-tetrahydro-1,8-naphthyridyl)-aminoethyloxy]-2-naphthaleneacetic Acid

A solution of ethyl 1,2,3,4-tetrahydro-6-[2-(5,6,7,8-tetrahydro-1,8-naphthyridyl)aminoethyloxy]-2-naphthaleneacetate (0.10 g) in ethanol was added sodium hydroxide (10%, 2.5 mL) and was stirred for 6 h. The reaction mixture was concentrated and the residue was purified by hplc to afford 0.80 g of the desired product. ¹H NMR δ 7.56 (d, 1H, J=7.4 Hz), 6.89 (d, 1H, J=8.3 Hz), 6.68 (d, 1H, J=7.3 Hz), 6.59-6.62 (m, 2H), 4.20 (t, 2H, J=5.9 Hz), 3.47 (t, 2H, J=5.6 Hz), 3.1 (t, 2H, J=5.9 Hz), 2.73-2.80 (m, 5H), 2.29-2.38 (m, 3H), 2.13 (m, 1H), 1.92 (m, 3H), 1.40 (m, 1H). Anal. Calcd for C₂₂H₂₆N₂O₃: Mol. Wt, 366.1943. Found: Mol. Wt, 366.1940 (HRMS).

The activity of the compounds of the present invention was tested in the following assays. Compounds of the present invention antagonize the αᵥβ₃ integrin with an IC₅₀ of 0.1 nM to 100 μM in the 293-cell assay. Similarly these compounds also antagonized the αᵥβ₅ integrin with an IC₅₀ of <50 μM in the cell adhesion assay.

### Vitronectin Adhesion Assay

Materials

Human vitronectin receptors αᵥβ₃ and αᵥβ₅ were purified from human placenta as previously described [Pytela et al., Methods in Enzymology, 144:475-489 (1987)]. Human vitronectin was purified from fresh frozen plasma as previously described [Yatohgo et al., Cell Structure and Function, 13:281-292 (1988)]. Biotinylated human vitronectin was prepared by coupling NHS-biotin from Pierce Chemical Company (Rockford, Ill.) to purified vitronectin as previously described [Charo et al., J. Biol. Chem., 266(3):1415-1421 (1991)]. Assay buffer, OPD substrate tablets, and RIA grade BSA were obtained from Sigma (St. Louis, Mo.). Anti-biotin antibody was obtained from Sigma (St. Luois, Mo.). Nalge Nunc-Immuno microtiter plates were obtained from Nalge Company (Rochester, N.Y.).

### Methods

### Solid Phase Receptor Assays

This assay was essentially the same as previously reported [Niiya et al., Blood, 70:475-483 (1987)]. The purified human vitronectin receptors αᵥβ₃ and αᵥβ₅ were diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptors were immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL was added to each well. After 45 minutes, the plates were washed and incubated with OPD/H₂O₂ substrate in 100 mM/L Citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCEL spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm [Rodbard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoiminomethyl)amino]-1-oxopentyl]amino]-1-oxoethyl]amino]-3-pyridinepropanoic acid [U.S. Pat. No. 5,602,155 Example 1] which is a potent αᵥβ₃ antagonist (IC₅₀ in the range 3-10 nM) was included on each plate as a positive control.

### Purified IIb/IIIa Receptor Assay

Materials

Human fibrinogen receptor (IIb/IIIa) was purified from outdated platelets. (Pytela, R., Pierschbacher, M. D., Argraves, S., Suzuki, S., and Rouslahti, E. “Arginine-Glycine-Aspartic acid adhesion receptors”, Methods in Enzymology 144(1987):475-489.) Human vitronectin was purified from fresh frozen plasma as described in Yatohgo, T., Izumi, M., Kashiwagi, H., and Hayashi, M., “Novel purification of vitronectin from human plasma by heparin affinity chromatography,” Cell Structure and Function 13(1988):281-292. Biotinylated human vitronectin was prepared by coupling NHS-biotin from Pierce Chemical Company (Rockford, Ill.) to purified vitronectin as previously described. (Charo, I. F., Nannizzi, L., Phillips, D. R., Hsu, M. A., Scarborough, R. M., “Inhibition of fibrinogen binding to GP IIb/IIIa by a GP IIIa peptide”, J. Biol. Chem. 266(3)(1991): 1415-1421.) Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from (Rochester, N.Y.). ADP reagent was obtained from Sigma (St. Louis, Mo.).

### Methods

Solid Phase Receptor Assays

This assay is essentially the same reported in Niiya, K., Hodson, E., Bader, R., Byers-Ward, V. Koziol, J. A., Plow, E. F. and Ruggeri, Z. M., “Increased surface expression of the membrane glycoprotein IIb/IIIa complex induced by platelet activation: Relationships to the binding of fibrinogen and platelet aggregation”, Blood 70(1987):475-483. The purified human fibrinogen receptor (IIb/IIIa) was diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptor was immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL were added to each well. After 45 minutes, the plates were washed and incubated with ODD/H₂O₂ substrate in 100 mM/L citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCELJ spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm, [Robard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoiminomethyl)amino]-1-oxopentyl]amino]-1-oxoethyl]amino]-3-pyridinepropanoic acid, bistrifluoroacetate salt [U.S. Pat. No. 5,602,155 Example 1] which is a potent IIb/IIIa antagonist (IC₅₀ in the range 8-18 nM) was included on each plate as a positive control.

### Human Platelet Rich Plasma Assays

Healthy aspirin free donors were selected from a pool of volunteers. The harvesting of platelet rich plasma and subsequent ADP induced platelet aggregation assays were performed as described in Zucker, M. B., “Platelet Aggregation Measured by the Photometric Method”, Methods in Enzymology 169(1989):117-133. Standard venipuncture techniques using a butterfly allowed the withdrawal of 45 mL of whole blood into a 60 mL syringe containing 5 mL of 3.8% trisodium citrate. Following thorough mixing in the syringe, the anti-coagulated whole blood was transferred to a 50 mL conical polyethylene tube. The blood was centrifuged at room temperature for 12 minutes at 200×g to sediment non-platelet cells. Platelet rich plasma was removed to a polyethylene tube and stored at room temperature until used. Platelet poor plasma was obtained from a second centrifugation of the remaining blood at 2000×g for 15 minutes. Platelet counts are typically 300,000 to 500,000 per microliter. Platelet rich plasma (0.45 mL) was aliquoted into siliconized cuvettes and stirred (1100 rpm) at 37° C. for 1 minute prior to adding 50 uL of pre-diluted test compound. After 1 minute of mixing, aggregation was initiated by the addition of 50 uL of 200 uM ADP. Aggregation was recorded for 3 minutes in a Payton dual channel aggregometer (Payton Scientific, Buffalo, N.Y.). The percent inhibition of maximal response (saline control) for a series of test compound dilutions was used to determine a dose response curve. All compounds were tested in duplicate and the concentration of half-maximal inhibition (IC₅₀) was calculated graphically from the dose response curve for those compounds which exhibited 50% or greater inhibition at the highest concentration tested; otherwise, the IC₅₀ is reported as being greater than the highest concentration tested.

What is claimed is:

1. A compound of the formula I 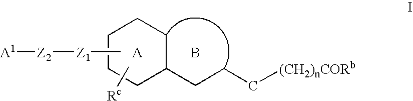
wherein
A¹ is a 5-9 membered monocyclic or 7-12 membered polycyclic heterocycle of the formula 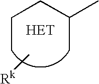
containing at least one nitrogen atom and 0 to 5 heteroatoms or groups selected from O, N, S, SO₂ or CO; optionally saturated or unsaturated; optionally substituted by one or more R^{k} selected from the group consisting of hydroxy, alkyl, alkoxy, alkoxy-alkyl, thioalkyl, haloalkyl, cyano, amino, alkylamino, halogen, acylamino, sulfonamide and —COR wherein R is hydroxy, alkoxy, alkyl or amino;
or A¹ is 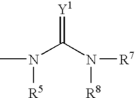
wherein Y¹ is selected from the group consisting of N—R², O, and S;
R² is selected from the group consisting of H; alkyl; aryl; hydroxy; alkoxy; cyano; alkenyl; alkynyl; amido; alkylcarbonyl; arylcarbonyl; alkoxycarbonyl; aryloxycarbonyl; haloalkylcarbonyl; haloalkoxycarbonyl; alkylthiocarbonyl; arylthiocarbonyl; acyloxymethoxycarbonyl;
R² taken together with R⁷ forms a 4-12 membered dinitrogen containing heterocycle optionally substituted with one or more substituent selected from the group consisting of lower alkyl, thioalkyl, alkylamino, hydroxy, keto, alkoxy, halo, phenyl, amino, carboxyl or carboxyl ester, and fused phenyl;
or R² taken together with R⁷ forms a 4-12 membered heterocycle containing one or more heteroatom selected from O, N and S optionally unsaturated;
or R² taken together with R⁷ forms a 5 membered heteroaromatic ring fused with a aryl or heteroaryl ring;
R⁷ (when not taken together with R²) and R⁸ are independently selected from the group consisting of H; alkyl; alkenyl; alkynyl; aralkyl; amino; alkylamino; hydroxy; alkoxy; arylamino; amido, alkylcarbonyl, arylcarbonyl; alkoxycarbonyl; aryloxy; aryloxycarbonyl; haloalkylcarbonyl; haloalkoxycarbonyl; alkylthiocarbonyl; arylthiocarbonyl; acyloxymethoxycarbonyl; cycloalkyl; bicycloalkyl; aryl; acyl; benzoyl;
or NR⁷ and R⁸ taken together form a 4-12 membered mononitrogen containing monocyclic or bicyclic ring optionally substituted with one or more substituent selected from lower alkyl, carboxyl derivatives, aryl or hydroxy and wherein said ring contains 0-1 heteroatom, selected from the group consisting of O, N and S;
R⁵ is selected from the group consisting of H, and alkyl;
or
A is 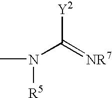
wherein Y² is selected from the group consisting of alkyl; cycloalkyl; bicycloalkyl; aryl; monocyclic heterocycles;
Z₁ is selected from the group consisting of CH₂, O, CH₂O, NH, CO, S, SO, CH(OH) and SO₂;
Z₂ is a 1-5 carbon linker optionally containing one or more heteroatom selected from the group consisting of O, S and N; alternatively Z₁-Z₂ may further contain a carboxamide, sulfone; sulfonamide, alkenyl, alkynyl, or acyl group; wherein the carbon and nitrogen atoms of Z₁-Z₂ are optionally substituted by alkyl, alkoxy, thioalkyl, alkylsulfone, aryl, alkoxyalkyl, alkylamino, heteroaryl, hydroxy, alkenyl, alkynyl, carboxyalkyl, halogen, haloalky or acylamino;
is an integer 0, 1 or 2;
R^{c} is selected from the group consisting of hydrogen; alkyl; halogen, hydroxy, nitro, alkoxy, amino, haloalkyl, aryl, heteroaryl, alkoxyalkyl, aminoalkyl, hydroxyalkyl, thioalkyl, alkylamino, arylamino, alkylsulfonylamino, acyl, acylamino, sulfonyl, sulfonamide, allyl, alkenyl, methylenedioxy, ethylenedioxy, alkynyl, alkynylalkyl, carboxy, alkoxycarbonyl, carboxamido, cyano, and —(CH₂)ₙ—COR wherein n is 0-2 and R is selected from hydroxy, alkoxy, alkyl and amino;
X is selected from the group consisting of —O—, CO, SO₂, NR^{m} and (CHR^{p})ₙ; wherein R^{p} and R^{m} are H or alkyl, n is 0-2;
R^{b} is X₃-R^{h} wherein X₃ is selected from the group consisting of O, S and NR^{j} wherein R^{h} and R^{j} are independently selected from the group consisting of H, alkyl, acyl, aryl, aralkyl and alkoxyalkyl; and
The ring A-B, 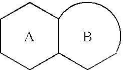
is selected from the group consisting of 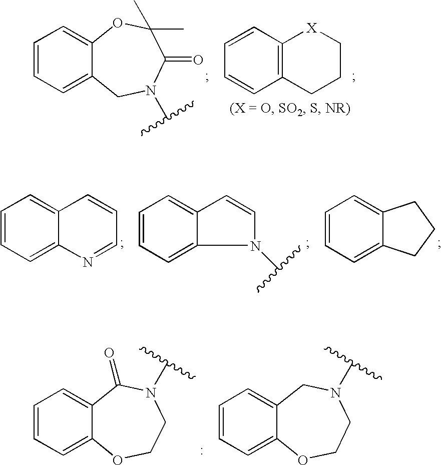
all optionally substituted and bonded to X and Z₁ at any position;
and pharmaceutically acceptable salts, isomers, enantiomers, tautomers, racemates and polymorphs thereof.

2. A compound according to claim 1 wherein 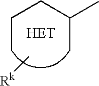
is selected from the group consisting of 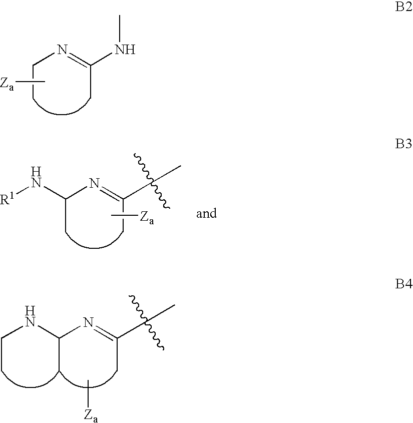
wherein Zₐ is H, alkyl, alkoxy, hydroxy, amine, alkylamine, dialkylamine, carboxyl, alkoxycarbonyl, hydroxyalkyl, halogen or haloalkyl and R¹ is H, alkyl, alkoxyalkyl, acyl, haloalkyl or alkoxycarbonyl, and pharmaceutically acceptable salts, isomers, enantiomers, tautomers, racemates and polymorphs thereof.

3. A compound according to claim 1 wherein 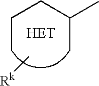
is selected from the group consisting of 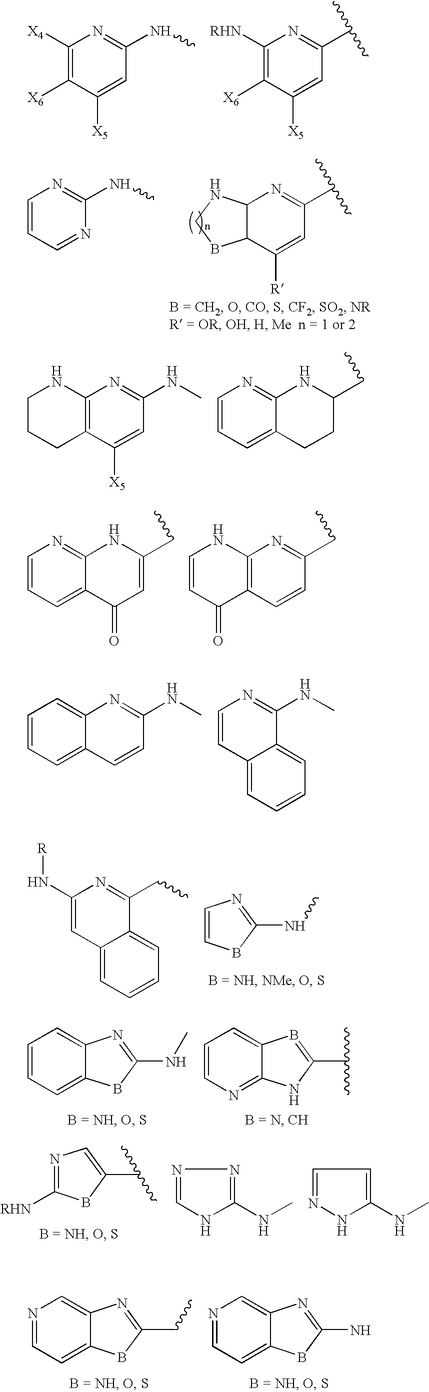 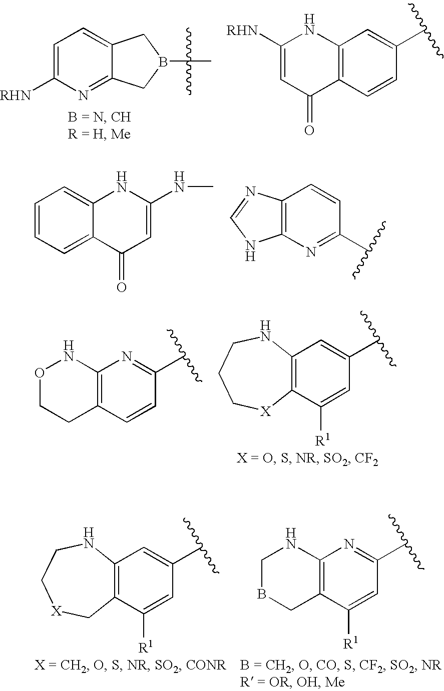
wherein X₄ and X₅ are selected from the group consisting of H, alkyl, branched alkyl, alkylamino, alkoxyalkylamino, haloalkyl, thioalkyl, halogen, amino, alkoxy, aryloxy, alkoxyalkyl, hydroxy, cyano, acylaminomethyl, methoxy, amine, methylamine, trifluoromethyl, dimethyl-amine, hydroxy, chloro, bromo, fluoro and cyano; X₆ is H, alkyl, hydroxy, halogen, alkoxy and haloalkyl; the pyridyl ring can be fused with a 4-8 membered ring, optionally saturated or unsaturated, and pharmaceutically acceptable salts, isomers, enantiomers, tautomers, racemates and polymorphs thereof.

4. A compound according to claim 1 wherein when Z₁ is CO or SO₂, and the linkage A¹-Z₂ is a heterocycle derived ring system selected from the group consisting of pyridine, imidazole, thiazole, oxazole, benzimidazole, and imidazopyridine, and pharmaceutically acceptable salts, isomers, enantiomers, tautomers, racemates and polymorphs thereof.

5. A compound according to claim 4 wherein the heterocycle derived ring systems for A¹-Z₂ are selected from the group consisting of: 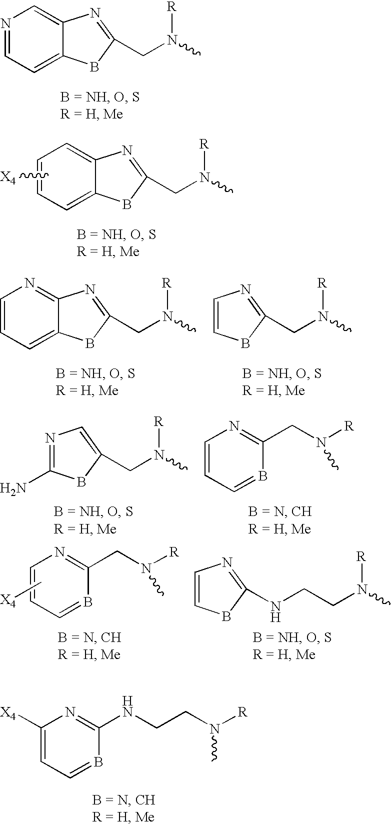
and pharmaceutically acceptable salts, isomers, enantiomers, tautomers, racemates and polymorphs thereof.

6. A compound according to claim 1 wherein the ring A-B 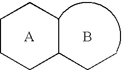
is a tetrahydronaphthalene 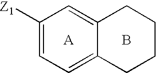
and Z₁ is S, and pharmaceutically acceptable salts, isomers, enantiomers, tautomers, racemates and polymorphs thereof.

7. A compound according to claim 1, wherein the ring A-B 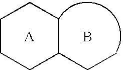
is a tetrahydronaphthalene 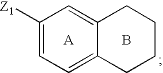
Z₁ is a CH₂;
A¹ is selected from the group consisting of: 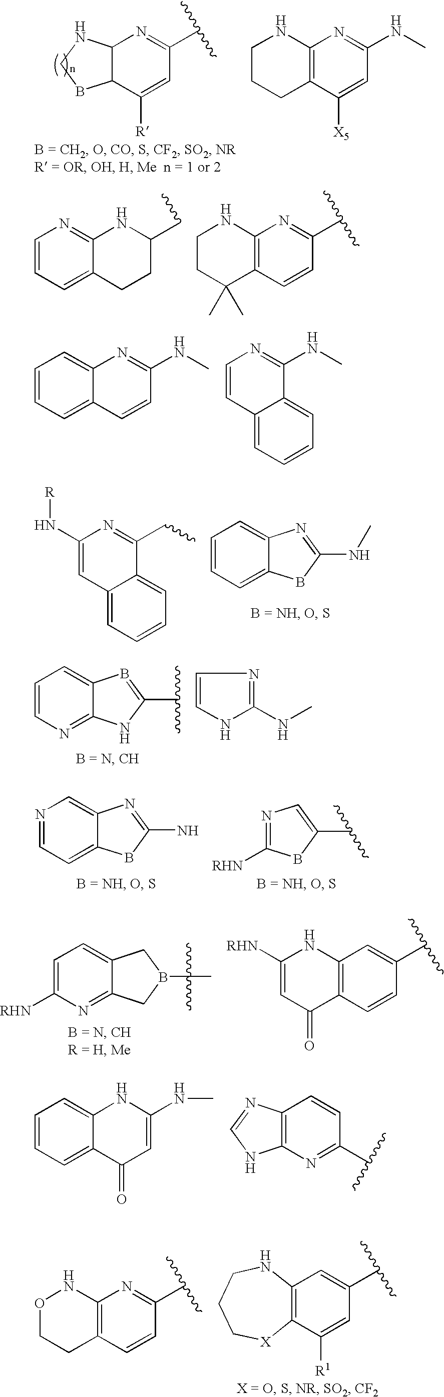 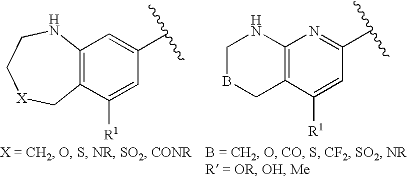
and pharmaceutically acceptable salts, isomers, enantiomers, tautomers, racemates and polymorphs thereof.

8. A compound according to claim 1, wherein
the ring A-B 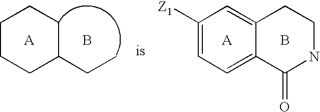
A¹ is selected from the group consisting of: 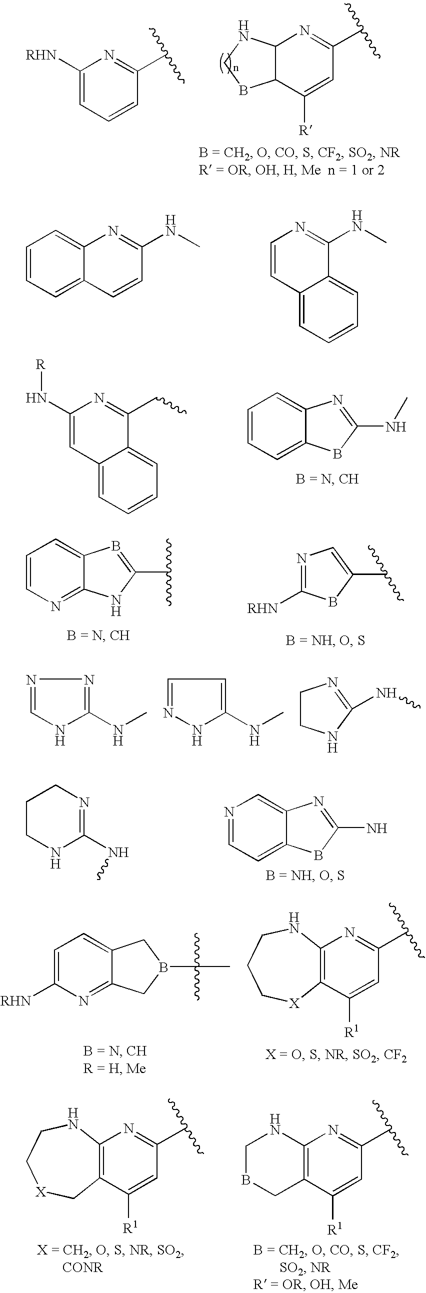
and pharmaceutically acceptable salts, isomers, enantiomers, tautomers, racemates and polymorphs thereof.

9. A compound according to claim 1 selected from the group consisting of:
[2,2-dimethyl-3-oxo-8-[3-(pyridin-2-ylamino)propoxy]-2,3-dihydro-1,4-benzoxazepin-4(5H)-yl]acetic acid;
1,2,3,4-tetrahydro-6-[3-(2-pyridinylamino)propoxy-2-isoquinoline-propanoic acid;
{5-[3-(pyridin-2-ylamino)propoxy]-1H-indol-1-yl}acetic acid; 2,3-dihydro-5-[3-(2-pyridinylamino)propoxy]-1H-indene-2-acetic acid;
2,3,4,5-tetrahydro-5-oxo-8-[3-(2-pyridinylamino)propoxy]-1,4-benzoxazepine-4-acetic acid;
2,3,4,5-tetrahydro-8-[3-(2-pyridinylamino)propoxy]1,4-benzoazepine-4-acetic acid;
1,2,3,4-tetrahydro-1-oxo-6-[3-(2-tetrahydropyrimidinyl)amino]-propoxy]-2-isoquinolineacetic acid;
3,4-dihydro-7-[3-(2-pyridinylamino)propoxy]-2-H-1-benzopyran-3-acetic acid;
(6-{[3-(pyridin-2-ylamino)propyl]thio}-1,2,3,4-tetrahydronaphthalen-2-yl)acetic acid;
1,2,3,4-tetrahydro-6-[2-(5,6,7,8-tetrahydro-1,8-naphthyridyl)-aminoethyl xy] 2-naphthaleneacetic acid, and pharmaceutically acceptable salts, isomers, enantiomers, tautomers, racemates and polymorphs thereof.

10. A pharmaceutical composition comprising a therapeutically effective amount of a compound according to any one of claims 1 to 9 and a pharmaceutically acceptable carrier.

11. The use of a compound according to any one of claims 1 to 9 for the manufacture of a pharmaceutical composition for treating conditions mediated by the αᵥβ₃ integrin in a mammal in need of such treatment.

12. A use according to claim 11, wherein the condition treated is selected from tumor metastasis, tumor growth, solid tumor growth, angiogenesis, osteoporosis, humoral hypercalcemia of malignancy, smooth muscle cell migration, restenosis, atheroscelorosis, macular degeneration, retinopathy, and arthritis.

13. The use of a compound according to any one of claims 1 to 9 for the manufacture of a pharmaceutical composition for treating conditions mediated by the αᵥβ₅ integrin in a mammal in need of such treatment.

14. A use according to claim 13, wherein the condition treated is selected from tumor metastasis, tumor growth, solid tumor growth, angiogenesis, osteoporosis, humoral hypercalcemia of malignancy, smooth muscle cell migration, restenosis, atheroscelorosis, macular degeneration, retinopathy, and arthritis.

15. The use of a compound according to any one of claims 1 to 9 for the manufacture of a pharmaceutical composition for treating neoplasia in a patient in need thereof, wherein the method comprises administration of the pharmaceutical composition to the patient in combination with a chemotherapeutic agent.
